(19) United States
(12) Patent Application Publication
(10) Pub. No.: US 2021/0327548 A1
(43) Pub. Date: Oct. 21, 2021

(54) STORING, AUTHENTICATING, AND TRANSMITTING HEALTH DATA

(71) Applicant: VACMOBILE CORPORATION, Wilmington, DE (US)

(72) Inventors: Jennifer M. Sparks, Alpharetta, GA (US); William Sparks, Alpharetta, GA (US); John Thielens, Milton, GA (US); Cindy Taylor, Marietta, GA (US); Ben Cagle, Alpharetta, GA (US)

(21) Appl. No.: 17/234,491

(22) Filed: Apr. 19, 2021

Related U.S. Application Data
(63) Continuation-in-part of application No. 16/921,784, filed on Jul. 6, 2020.
(60) Provisional application No. 63/157,102, filed on Mar. 5, 2021.
(60) Provisional application No. 63/030,768, filed on May 27, 2020.
(60) Provisional application No. 63/011,767, filed on Apr. 17, 2020.

(51) Int. Cl. G16H 10/60; G06F 21/31; G06F 21/62; G16H 20/13; G16H 40/20; G06K 19/06; G06K 7/14

(52) U.S. Cl. CPC G16H 10/60; G06F 21/31; G06F 21/6245; G06K 7/1417; G16H 40/20; G06K 19/06037; G16H 20/13

(57) ABSTRACT

A computing device can authenticate a user account associated with a user. The computing device can scan an area to determine a scannable identifier associated with a vaccination dose. The computing device can determine a batch number and a lot number associated with the vaccination dose based on the scannable identifier. The computing device can determine a location associated with the at least one computing device. The computing device can receive a provider confirmation code corresponding to a provider. The computing device can perform a verification that the provider confirmation code matches an assigned provider confirmation code corresponding to the provider. The computing device can associate the vaccination dose with the user account in the data store as having been given to the user.

### CROSS-REFERENCE TO RELATED APPLICATIONS

This application is a continuation-in-part application of U.S. Non-Provisional patent application Ser. No. 16/921,784, filed Jul. 6, 2020, and entitled “SYSTEMS AND METHODS FOR STORING, AUTHENTICATING, AND TRANSMITTING DIGITAL HEALTH INFORMATION AND RECORDS,” and claims priority to U.S. Provisional Patent Application No. 63/157,102, filed Mar. 5, 2021, and entitled “SYSTEMS AND METHODS FOR STORING, AUTHENTICATING, AND TRANSMITTING DIGITAL HEALTH,” U.S. Provisional Patent Application No. 63/030,768, filed May 27, 2020, and entitled “SYSTEMS AND METHODS FOR STORING, AUTHENTICATING AND TRANSMITTING DIGITAL HEALTH INFORMATION AND RECORDS,” and U.S. Provisional Patent Application No. 63/011,767, filed Apr. 17, 2020, and entitled “SYSTEMS AND METHODS FOR STORING, AUTHENTICATING AND TRANSMITTING DIGITAL HEALTH INFORMATION AND RECORDS,” and the disclosures of which are herein incorporated by reference in their entireties.

### TECHNICAL FIELD

Embodiments of the present invention pertain to systems and methods for storing, authenticating and transmitting digital health information and records.

### BACKGROUND OF THE INVENTION

A state immunization registry (SIR), also known as an immunization information system, is a computer system that has information on the shots that have been given to an individual. SIRs are different in every state, but most contain at least the information such as: patient name (first, middle, and last), patient birth date, patient sex, patient birth state/country, mother's name, the types and dates of vaccines given, and the date the shot was given. However, no federal system tracks vaccination information of individuals across states. Additionally, SIRs are limited with respect to sharing by federal laws that protect a user's privacy. Individuals also do not generally have direct access to the SIRs so they cannot use an SIR to provide proof of being vaccinated except to organizations that have access to the SIR (e.g., physicians and hospitals). Therefore, there is a long-felt but unresolved need for systems and methods that provide accurately provide vaccination information for use by individuals and organizations.

### SUMMARY OF THE INVENTION

A primary use of SIRs is to ensure that children have all of their immunizations and that the information is available when your doctor needs it. All of the immunizations given are based on a schedule that tells providers when certain shots should be given. The immunization schedule is confusing and sometimes changes. SIRs can be used to help doctors decide which shots should be given and when.

SIRs simplify immunization record keeping, provide quicker access to immunization records, and help keep track of a patient's immunization status. SIRs can also help medical professionals locate immunization information for children new to their practice, provide official copies of immunization records, reduce or eliminate chart pulls needed for coverage assessment and Healthcare Effectiveness Data and Information Set (HEDIS) reviews, and facilitate routine doses administered reports for vaccine accountability.

Unfortunately, no national organization maintains vaccination records or information. Such immunization records can therefore be difficult to locate. For example, individuals who have lived in a multiplicity of states will need to contact those SIRs and/or medical professionals in each respective state to obtain their vaccination records. Such records are often needed, for example, for entry into child-care, kindergarten, school, summer camp, college, employment and/or international travel.

SIRs often have different protocols for individuals to request a copy of their vaccination records. The registries are not connected so if you have lived in more than one State you must contact more than one registry and follow their instructions. None of these processes are real time and unlike the systems and methods of embodiments of the invention, individuals need to make a separate request any time they wish to obtain a new copy of their records. None of SIRs offer the option to have the records sent to a third-party requester, and none of the SIRs offer users an option to have a QR code generated from their mobile phone that will provide them with a digital certificate of immunization and COVID-19 health status records.

Also, when visiting a new doctor or other medical professional, vaccination records may need to be provided to obtain appropriate medical treatment or care. For example, if immunization records are provided to you, are not provided to a SIR, are lost or are otherwise not available, you or your child may have to repeat vaccination doses or receive vaccinations that would otherwise not be needed if vaccination records were available. Patching together old immunization records from a number of years and across a number of states can be a daunting and time-consuming task. Failure to maintain accurate vaccination records can pose a serious health threat to the individual and the public.

It is an object of embodiments of the present invention to provide systems and methods for storing, retrieving and electronically providing individual vaccination information and records that are complete, accurate and certified by one or more SIRs, to the user and/or a third party.

It is another object of embodiments of the present invention to consolidate user vaccination records, verify vaccination requirements with organizations such as schools or employers, and electronically forward certified vaccination records to such entities by the user or automatically and directly to such entities by the system.

It is another object of embodiments of the present invention to confidentially and securely provide users with their consolidated vaccination records, from a multiplicity of sources, on electronic devices such as a desktop computer, a laptop computer, a mobile device and/or a tablet, as specified by the user.

It is another object of embodiments of the present invention to provide up-to-date reminders to users and/or their dependents so they are aware of relevant, suggested and/or required vaccines by federal, state, local, organizational, and the Centers for Disease Control and Prevention vaccination recommendations, guidelines, regulations, and laws.

It is another object of embodiments of the present invention to extend the aforementioned objects of the invention to vaccination records of non-humans, such as user pets and other animals.

It is another object of embodiments of the present invention to electronically provide individual pet/animal vaccination information and records that are certified, for example, by a veterinarian, to an entity such as an animal boarding facility, care facility, or kennel.

An electronic health records system includes a processor and a tangible, non-transitory medium configured to communicate with the processor. The memory has instructions stored thereon that, in response to execution by the processor, causes the system to perform operations that include i) allowing a user to register with the electronic health records system by entering identification information and medical information; ii) authorizing a user to access the electronic health records system subsequent to registering; and iii) obtaining, responsive to a request from the user, electronic immunization records for the user from an immunization registry of a first state.

In another embodiment, an electronic health records system includes a processor, and a tangible, non-transitory medium configured to communicate with the processor. The memory has instructions stored thereon that, in response to execution by the processor, cause the system to perform operations including: i) allowing a user to register with the electronic health records system by entering identification information and medical information; ii) authorizing a user to access and obtain the electronic health records system subsequent to registering; and iii) obtaining, responsive to a request from the user, COVID-19 health status records for the user from at least one of an immunization registry of a first state and an FDA approved bar coded in-home test. It should be understood that in addition to COVID-19 health status records, the system may obtain and transmit health status records for the user for any communicable disease for which a test or vaccine can be used to protect the health of the user or subscriber.

According to a first aspect, a system, comprising: A) a data store; and B) at least one computing device in communication with the data store, the at least one computing device configured to: 1) authenticate a user account associated with a user; 2) scan an area to determine a scannable identifier associated with a vaccination dose; 3) determine a batch number and a lot number associated with the vaccination dose based on the scannable identifier; 4) determine a location associated with the at least one computing device; 5) receive a provider confirmation code corresponding to a provider; 6) perform a verification that the provider confirmation code matches an assigned provider confirmation code corresponding to the provider, the location corresponds to a business location of the provider, and the batch number and the lot number associated with the vaccination dose are assigned to the provider; 7) and in response to the verification being successfully, associate the vaccination dose with the user account in the data store as having been given to the user.

According to a further aspect, the system of the first aspect or any other aspect, wherein the at least one computing device is further configured to determine an appointment time of the user with the provider. According to a further aspect, the system of the first aspect or any other aspect, wherein the provider confirmation code is further associated with the business location of the provider and a plurality of different provider confirmation codes are individually associated with a respective one of a plurality of other business locations of the provider. According to a further aspect, the system of the first aspect or any other aspect, wherein the at least one computing device is further configured to: A) request vaccination information associated with the vaccination dose from a third-party system based on the batch number and the lot number; and B) receive the vaccination information from the third-party system, the vaccination information. According to a further aspect, the system of the first aspect or any other aspect, wherein the vaccination information from the third-party system comprises a time corresponding to when the vaccination dose was dispensed. According to a further aspect, the system of the first aspect or any other aspect, wherein the area scanned to determine the scannable identifier comprises a vile of the vaccination dose.

According to a second aspect, a method, comprising: A) scanning, via at least one computing device, a vial of a vaccination dose to determine a scannable identifier associated with the vaccination dose; B) determining, via the at least one computing device, a batch number and a lot number associated with the vaccination dose based on the scannable identifier; C) determining, via the at least one computing device, a location associated with the at least one computing device; D) receiving, via the at least one computing device, a provider confirmation code corresponding to a provider; E) performing, via the at least one computing device, a verification that the provider confirmation code matches an assigned provider confirmation code corresponding to the provider, the location corresponds to a business location of the provider, and the batch number and the lot number associated with the vaccination dose are assigned to the provider; and F) in response to the verification being successfully, associating, via the at least one computing device, the vaccination dose with a user account as having been given to a user corresponding to the user account.

According to a further aspect, the method of the second aspect or any other aspect, wherein associating the vaccination dose with the user account comprises: A) generating a record stored locally on the at least one computing device, the record comprising information about the vaccination dose and the user account; and B) transmitting the record to a remote data service for storage. According to a further aspect, the method of the second aspect or any other aspect, further comprising: A) generating, via the at least one computing device, a second scannable identifier encoding information regarding the record; and B) rendering, via the at least one computing device, the second scannable identifier on a display. According to a further aspect, the method of the second aspect or any other aspect, wherein the second scannable identifier comprises a link to a user interface comprising a certificate of immunization for the vaccination dose. According to a further aspect, the method of the second aspect or any other aspect, further comprising: A) converting, via the at least one computing device, the record into a predetermined format associated with a third-party; and B) pushing, via the at least one computing device, the record in the predetermined format to the third-party.

According to a further aspect, the method of the second aspect or any other aspect, further comprising: A) requesting, via the at least one computing device, vaccination information associated with the vaccination dose from a third-party system based on the batch number and the lot number; and B) receiving, via the at least one computing device, the vaccination information from the third-party system. According to a further aspect, the method of the second aspect or any other aspect, wherein the vaccination information from the third-party system comprises the assigned provider confirmation code corresponding to the provider.

According to a third aspect, A non-transitory computer-readable medium embodying a program that, when executed by at least one computing device, causes the at least one computing device to: A) scan a vial of a vaccination dose to determine a scannable identifier associated with the vaccination dose; B) determine a batch number and a lot number associated with the vaccination dose based on the scannable identifier; C) determine a location associated with the at least one computing device; D) receive a provider confirmation code corresponding to a provider; E) perform a verification that the provider confirmation code matches an assigned provider confirmation code corresponding to the provider, the location corresponds to a business location of the provider, and the batch number and the lot number associated with the vaccination dose are assigned to the provider; and F) generate a record comprising information about the vaccination dose and a user account associated with a user, the record indicating that the user has been given the vaccination dose.

According to a further aspect, the non-transitory computer readable medium of the third aspect or any other aspect, wherein the program further causes the at least one computing device to: A) request vaccination information associated with the vaccination dose from a third-party system based on the batch number and the lot number; and B) receive the vaccination information from the third-party system. According to a further aspect, the non-transitory computer readable medium of the third aspect or any other aspect, wherein the vaccination information from the third-party system comprises the business location of the provider. According to a further aspect, the non-transitory computer readable medium of the third aspect or any other aspect, wherein the program further causes the at least one computing device to transfer the record indicating that the user has been given the vaccination dose to a remote device.

According to a further aspect, the non-transitory computer readable medium of the third aspect or any other aspect, wherein the remote device comprises an electronic medical records (EMR) system of a healthcare provider. According to a further aspect, the non-transitory computer readable medium of the third aspect or any other aspect, wherein the record is transferred wirelessly from the at least one computing device using at least one of near field communication (NFC) or Bluetooth. According to a further aspect, the non-transitory computer readable medium of the third aspect or any other aspect, wherein the record comprises a digital signature generated by a particular certificate authority.

According to a fourth aspect, an electronic health record system, comprising: A) a processor; B) a tangible, non-transitory medium configured to communicate with the processor, the memory having instructions stored thereon that, in response to execution by the processor, cause the electronic health records system to perform operations comprising: C) receiving from a user identification information and medical information to register with the electronic health records system; D) authorizing a user to access the electronic health records system subsequent to registering; and E) obtaining, responsive to a request from the user, electronic immunization records for the user from an immunization registry of a first state.

According to a further aspect, the electronic health record system of the fourth aspect or any other aspect, further comprising: A) obtaining, responsive to a request from the user, electronic immunization records for the user from an immunization registry of a second state; and B) providing to the user, within the electronic health records system, combined electronic immunization records comprising the electronic immunization records from the first state and the electronic immunization records from the second state. According to a further aspect, the electronic health record system of the fourth aspect or any other aspect, the operations further comprising: A) transmitting, from the electronic health records system, responsive to a request from the user, at least a portion of the electronic immunization records to a third-party system.

According to a further aspect, the electronic health record system of the fourth aspect or any other aspect, the operations further comprising: A) transmitting, from the electronic health records system, responsive to a request from the user, at least a portion of the combined electronic immunization records to an external records recipient. According to a further aspect, the electronic health record system of the fourth aspect or any other aspect, the operations further comprising: A) providing, to the user and within the electronic health records system, access to immunization information for a relative of the user. According to a further aspect, the electronic health record system of the fourth aspect or any other aspect, the operations further comprising: A) receiving from the immunization registry a certificate of immunization indicating that the relative of the user is in compliance with immunization required by the state.

According to a further aspect, the electronic health record system of the fourth aspect or any other aspect, the operations further comprising: A) providing to the immunization registry of the first state, by the electronic health records system and responsive to a request from the user, the electronic immunization records obtained from the immunization registry of the second state. According to a further aspect, the electronic health record system of the fourth aspect or any other aspect, the operations further comprising: A) a registry adapter that receives data for the user, from a registry database of the first state, to complete forms required by the immunization registry of the first state. According to a further aspect, the electronic health record system of the fourth aspect or any other aspect, the operations further comprising: A) receiving from the immunization registry a state recognized certificate of immunization indicating that the user is in compliance with immunization required by the state in accordance with applicable laws and regulations.

According to a further aspect, the electronic health record system of the fourth aspect or any other aspect, the operations further comprising: A) providing to the user a QR code indicating that the user is in compliance with immunization required by the second state. According to a further aspect, the electronic health record system of the fourth aspect or any other aspect, wherein obtaining electronic immunization records comprises transmitting to the user, electronic immunization records from the first state. According to a further aspect, the electronic health record system of the fourth aspect or any other aspect, wherein a first responder enters an emergency code in a mobile device of the user to access and review the user's immunizations records. According to a further aspect, the electronic health record system of the fourth aspect or any other aspect, wherein the system communicates with software of the third-party system to transmit the user's immunization records to the third-party system. According to a further aspect, the electronic health record system of the fourth aspect or any other aspect, wherein the system receives health meta data for Countries, States, territories, Nations, Tribes, Pueblos, Counties, Cities and other municipalities.

According to a fifth aspect, an electronic health record system, comprising: A) a processor; B) a tangible, non-transitory medium configured to communicate with the processor, the memory having instructions stored thereon that, in response to execution by the processor, cause the electronic health records system to perform operations comprising: 1) receiving from a user identification information and medical information to register with the electronic health records system; 2) authorizing a user to access the electronic health records system subsequent to registering; and 3) obtaining, responsive to a request from the user, electronic COVID-19 health status records for the user from at least one of an immunization registry of a first state and a result from a bar coded in-home test analyzed by a third party.

According to a further aspect, the electronic health record system of the fifth aspect or any other aspect, further comprising: A) obtaining, responsive to a request from the user, COVID-19 health status records for the user from an immunization registry of a second state; and B) providing to the user, by the electronic health status records system, combined COVID-19 health status records comprising the COVID-19 health status records from the first state and the COVID-19 health status records from the second state. According to a further aspect, the electronic health record system of the fifth aspect or any other aspect, the operations further comprising: A) transmitting, from the electronic health records system, responsive to a request from the user, at least a portion of the COVID-19 health status records to a third-party system.

According to a further aspect, the electronic health record system of the fifth aspect or any other aspect, the operations further comprising: A) transmitting, to the user by the electronic health records system, COVID-19 health status records for a relative of the user. According to a further aspect, the electronic health record system of the fifth aspect or any other aspect, the operations further comprising: A) transmitting, to the user by the electronic health records system, a notification indicating that the relative of the user has tested negative for COVID-19. According to a further aspect, the electronic health record system of the fifth aspect or any other aspect, the operations further comprising: A) providing to the immunization registry of the first state, by the electronic health records system and responsive to a request from the user, the COVID-19 health status records obtained from the immunization registry of the second state.

According to a further aspect, the electronic health record system of the fifth aspect or any other aspect, the operations further comprising: A) a registry adapter that receives data for the user, from a registry database of the first state, to complete forms required by the immunization registry of the first state. According to a further aspect, the electronic health record system of the fifth aspect or any other aspect, the operations further comprising: A) providing, from the electronic health records system to the user, an indication that a relative of the user has tested negative for COVID-19. According to a further aspect, the electronic health record system of the fifth aspect or any other aspect, wherein the electronic health records system receives the indication from the immunization registry.

According to a further aspect, the electronic health record system of the fifth aspect or any other aspect, the operations further comprising: A) providing to the user a QR code indicating that the user has tested negative for COVID-19. According to a further aspect, the electronic health record system of the fifth aspect or any other aspect, wherein obtaining electronic COVID-19 health status records comprises transmitting to the user, COVID-19 health status records from the first state. According to a further aspect, the electronic health record system of the fifth aspect or any other aspect, wherein the system communicates with software of the third party system to transmit the user's electronic COVID-19 health status records to the third party system. According to a further aspect, the electronic health record system of the fifth aspect or any other aspect, wherein the system receives health meta data for Countries, States, territories, Nations, Tribes, Pueblos, Counties, Cities and other municipalities. According to a further aspect, the electronic health record system of the fifth aspect or any other aspect, wherein the bar coded in-home test is FDA approved.

### BRIEF DESCRIPTION OF THE DRAWINGS

FIG. 1 is a block diagram of an embodiment of a vaccination record verification system in accordance with an embodiment of the invention.

FIG. 2 illustrates a block diagram of the internal hardware of the computer and servers that can be used in the network and cloud shown in FIG. 1.

FIG. 3 shows a logical representation of the registry adapter shown in FIG. 1.

FIG. 4 is a screen shot showing a HIPAA and terms of user interface, for use with the system of FIG. 1.

FIG. 5 is a screen shot showing a user home page showing the various tasks the app can perform for the user.

FIG. 6 is a screen shot showing an account profile.

FIG. 7 is a screen shot that is shown when the vault tab is selected in FIG. 6.

FIG. 8 is a screen shot of a member's profile.

FIG. 9 is a screen shot that appears when the view full certificate of immunization icon is selected in FIG. 8.

FIG. 10 is a screen shot showing a full certificate of immunization when either a QR code is scanned or when the user wishes to see a print preview of the certificate of immunization.

FIG. 11 is a screen shot similar to that of FIG. 5 and includes push notifications for required and recommended vaccines and notifications when new information has been added to the member's vault.

FIG. 12 is a screen shot indicating that the subscriber has been registered for COVID-19 testing.

FIG. 13 is a screen shot that allows the subscriber and/or profile members to select a COVID-19 test.

FIG. 14 is a screen shot that allows COVID-19 enrolled subscriber or profile member to commence COVID-19 testing.

FIG. 15 is a screen shot that commences the COVID-19 testing process.

FIG. 16 is a screen shot that allows the uploading of a video of the COVID-19 testing process.

FIG. 17 is a screen shot indicating that the COVID-19 test of FIG. 16 has been registered with a COVID-19 testing facility.

FIG. 18 is a screen shot of a subscriber receiving a push notification that test results are ready to view in the vault, similar to that of FIG. 5.

FIG. 19 is a screen shot that includes a message indicating that the subscriber's COVID-19 test results have come back positive.

FIG. 20 is a screen shot that includes a message indicating that the subscriber's COVID-19 test results have come back negative.

FIG. 21 is a screen shot indicating that all members of a household have tested negative.

FIG. 22 is a screen shot that provides the COVID-19 health status of the subscriber.

FIG. 23 is a screen shot of the member's complete history of COVID-19 health status records.

FIG. 24 is a screen shot that allows the subscriber or a profile member to select a specific type of QR code.

FIG. 25 is a screen shot of a healthcare QR code that is similar to that of FIG. 23.

FIG. 26 is a screen shot showing subscription details for the subscriber.

FIG. 27 is a screen shot that shows exemplary user profile information required by state immunization registries for an individual to obtain their immunization records.

FIG. 28 shows a schematic representation of a user interface that allows an application to be downloaded, for use with the system of FIG. 1.

FIG. 29 shows an exemplary list of entities to which subscriber vaccination records can be sent.

FIG. 30 shows the use of a device that has a companion app that is used by a third party to read a subscriber's QR code such as generated in FIGS. 9 and/or 25, and/or a subscriber's biometric data to obtain access to the subscriber's immunization records and/or COVID-19 status.

FIG. 31 shows the use a free-standing device that has a companion app that is used to read a subscriber's QR code such as generated in FIGS. 9 and/or 25, and/or a subscriber's biometric data to obtain access to the subscriber's immunization records and/or COVID-19 status.

FIG. 32 shows a diagram that illustrates exemplary and non-limiting factors that determine when health alert trigger factors can be transmitted.

FIG. 33 shows exemplary health metrics that can be used to determine if a tele-health appointment can be made.

FIG. 34 shows an exemplary flowchart illustrating a process for authenticating a user's vaccination history.

### DETAILED DESCRIPTION OF THE INVENTION

While this invention is susceptible of embodiment in many different forms, there is shown in the drawings and described herein a preferred embodiment of the invention, with the understanding that the present disclosure is to be considered as an exemplification of the principles of the invention and is not intended to limit the broad aspect of the invention to the embodiment illustrated.

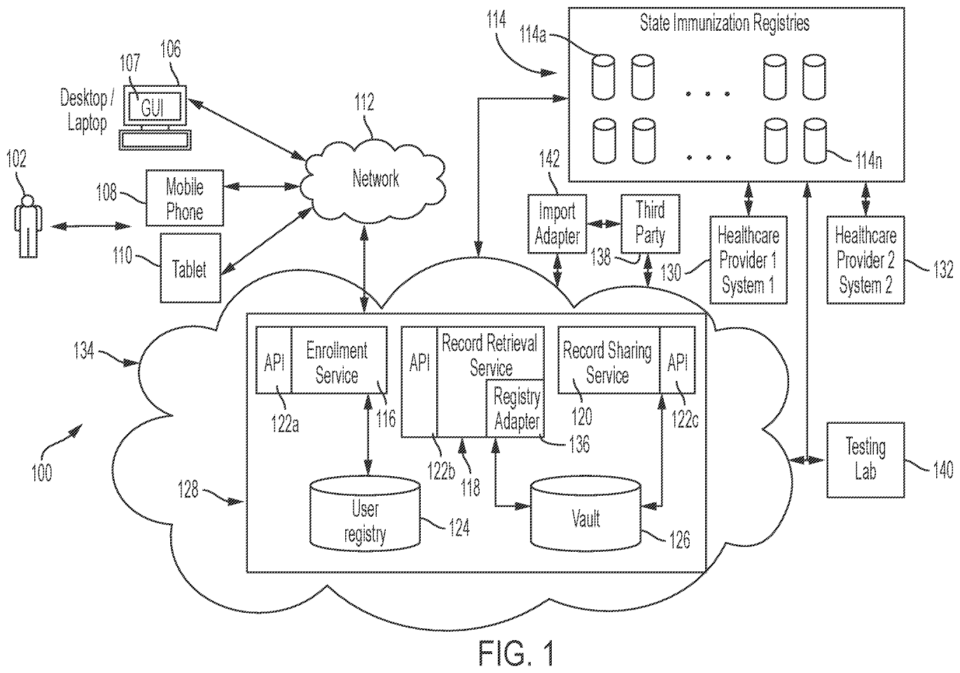

FIG. 1 is a block diagram of an embodiment of a vaccination record verification system 100 in accordance with an embodiment of the invention. The system 100 advantageously allows users to obtain, for example, copies of their immunization and COVID-19 records from their mobile phones.

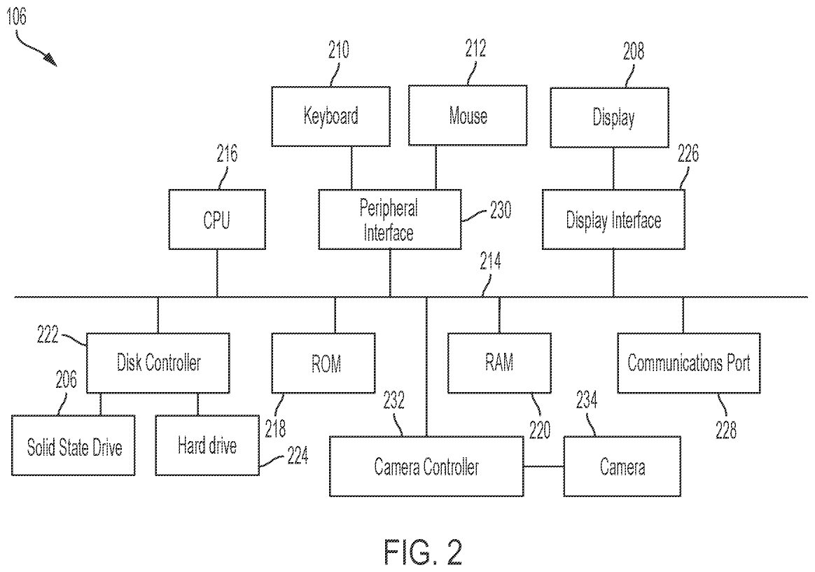

An exemplary computer-based system 100 for implementing a vaccination record verification embodiment of the invention allows a user 102 to interface with a graphical user interface (GUI) 107 that may be embodied on any number of computing devices, such as a mobile phone 108, desktop or laptop computer 106, and/or tablet computer 110. The GUI 107 generally operates under control of software read from a computer readable medium such as a conventional readable only memory (ROM) 218, a magnetic hard disk drive 224 and/or solid state drive 206, as shown in FIG. 2, which incorporates network protocols that communicate over a Wide Area Network (WAN) 112 such as the Internet. In turn, the network 112 can include one or more servers that includes a memory, network interface, and processor (not shown) that directly or indirectly executes or interfaces with various software components/software modules to communicate with a mobile phone 108, desktop or laptop computer 106, tablet computer 110 and vaccination record system 128.

As used herein a software component or module generally refers to a software application (or portion thereof), routine, API, program, script, data structure, or other memory resident application suitable for performing a specific task or responding to a certain command, signal or instruction. In one embodiment, the software components receive data relating, for example, to a physical scenario in the real world such as, for example, a user 102 receiving a new vaccination and/or a new COVID-19 test result. Further, this user 102 related data is transformed by the relevant software components to produce reports, guidance, recommendations, or other tangible output to help the user 102 remain informed about vaccination and/or COVID-19 health status.

Vaccination record system 128 includes user registry database 124 and vault database 126 that allow a user 102 to use a GUI 107 to input user enrollment information into registry database 124 and to retrieve and send to third parties 138 Health Insurance Portability and Accountability Act (HIPAA) compliant vaccination records and/or COVID-19 test results from vault database 126.

User registry database 124 and vault database 126 can be physically separate databases or separate logical areas of the same physical database. The user registry 124 is subject to normal software as a service (SaaS) identity regulations such as General Data Protection Regulation (GDPR) and California Consumer Privacy Act (CCPA) like any and retail web property. However, vault 126 contains personal health information (PHI) in it and will be subject to more stringent requirements and compliant with the Health Insurance Portability and Accountability Act (HIPAA), particular state requirements, such as the Federal Risk and Authorization Management Program (FedRAIVIP) for the state of Georgia (and other states), for example. Different implementations of the user registry database 124 and vault database 126 may be utilized in view of varying requirements. Any data in the vault database 126 pertains to some member in the user registry 124. As the user registry 124 is populated over time, relationships between users 102 and dependents, for example, can be used to create and enforce access policies on data stored in the vault 126. Google Cloud Services and Amazon Web Services offer and can be utilized in connection with HIPAA-compliant storage.

In one embodiment, a camera of the desktop/laptop 106, mobile phone 108 or tablet 110 can advantageously take a photo of a vaccination record from the doctor and automatically create a vaccination record and store the photo as a backup. As used herein, an “app” is short for “application,” which is a software program. While the term “app” typically refers to software programs for mobile phones 108 and tablets 110, the term “app” as used herein also includes software programs for any hardware platform, such as desktop/laptop computer 106.

The app can build a personalized immunization record for the user 102, the user's 102 children and/or the user's 102 pets. The primary features of system 100 can include HIPAA compliant cloud storage 134 that provides backup and recovery of photos of vaccine and/or COVID-19 records in vault 126, and optical character recognition to automatically create vaccination records from photos of doctor vaccination records or vaccination records of other medical professionals in a manner compliant with individual SIRs 114a- n.

The app provides a record entirely from a single photo: reading, for example, the name, date, vaccine administered. The app automatically creates a vaccination record that matches the requirements for the school/hospital asking for the records. Additional features can include location-based and objective-based customized forms that show the required vaccinations have been met to comply with federal, state, local, organizational, and Centers for Disease Control and Prevention vaccination recommendations, guidelines, regulations, and laws.

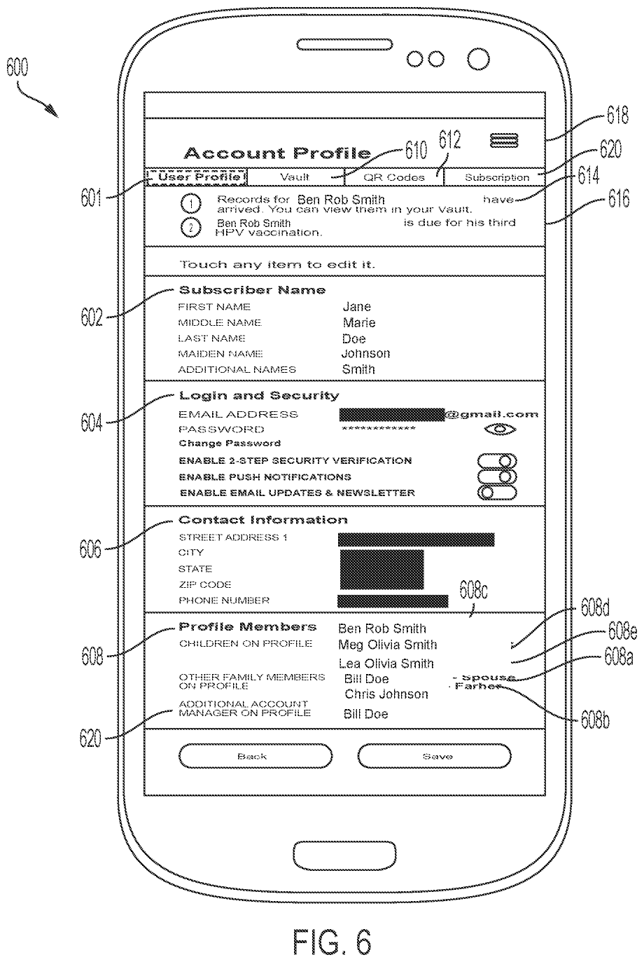

In an embodiment, the vaccination record system 128 can cause a screen display to appear on a mobile device 108 that recommends vaccines for the user 102, the user's 102 children, and/or the user's pet(s). For example, a screen display could be presented that asks: “Are you entering first grade this year? I will create a personalized plan that shows all your relevant vaccinations through elementary school.” The vaccination record system 128 can include, for example, a recommendation engine (not shown) that can provide push notifications that will alert the user 102 or the user's dependents 104 (and the subscriber 602 and/or profile members 608a-e in FIG. 6) when a required or recommended vaccination is due.

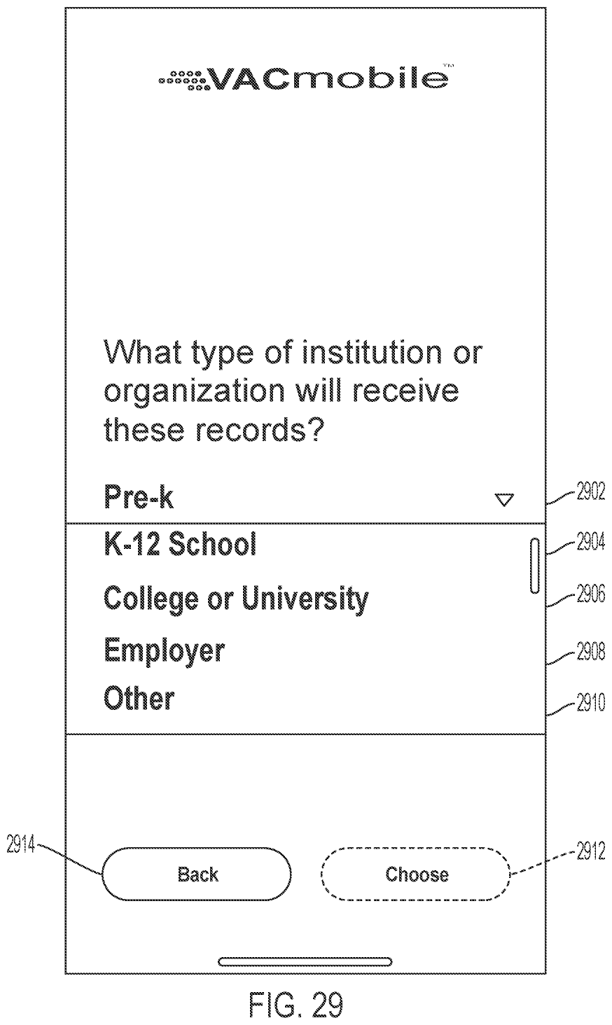

As shown in FIG. 29, the system 100 can also cause a screen display to appear on a mobile device 108 that asks a user 102 ifs/he would like to have a copy of the vaccination record electronically (or otherwise) transmitted to a third party 138, such as a K-12 school 2902, a college or university 2904, an employer 2906 or some other 2910 organization or entity, and verify the requirements are met on your behalf. To transmit a copy of the vaccination record, the user 102 selects “Choose” icon 2912; selection icon 2914 returns the user 102 to the previous screen. FIG. 29 can also include an option for the user 102 to pay a fee to have a copy of the vaccination record electronically (or otherwise) transmitted to a third party 138.

The vaccination record system 128 tracks and records each step or event during the certification process. For example, system 128 can provide to the user 102 and/or dependents 104 updates on steps or events such as “records printed,” “records mailed,” records received,” records verified,” and “process complete.” Each step or event would be recorded in the system 128, and an alert, notification or email would be transmitted to the user's 102 mobile phone 108, desktop or laptop computer 106, or tablet computer 110. The system 128 can also transmit an alert, notification or email to the user 102 when, for example, a booster for an existing vaccine and/or new vaccination is added to existing government requirements or recommendations.

In one embodiment, APIs 122a-c reside on the server, along with a software platform or engine, such as Amazon Web Services (AWS) or Microsoft ASP.NET. Various system and method embodiments described can use web browser-based AWS or ASP.NET software systems. In one embodiment, a server (not shown) in network 112 is accessed by a mobile phone 108, desktop or laptop computer 106 or tablet computer 110. The server in network 112 can exchange information with the vaccination record system 128 that includes user registry database 122 and vault database 124.

With the user's 102 permission, the vaccination record system 128 will query the SIRs 114a-n where you or your minor children have received vaccinations. Generally, immunization registries are a secure health information system that contain immunization records for persons living in a particular state in the U.S.A., for example. SIRs 114a-n for any particular state or jurisdiction are generally secure and confidential and include and store immunization records from multiple healthcare providers 130, 132 or other similar sources in one centralized system.

The import adapter 142 addresses a bulk automated use case where, for example, a school, for example, needs to collects data from a group of users 102 who have pre-authorized its release, but in a format that allows the data to be easily imported, for example, into a School Information System (SIS). A SIS can also be referred to as a Student Management System (SMS), Student Information Management System (SIMS) or Student Record System (SRS).

A SIS is a known tool that schools can use to manage student records and administrative matters. Generally, a SIS is a software solution that is designed to assist schools track and manage student data that pertains, for example, to grades, attendance, and behavioral. A SIS can be used as the primary database for school operations, and can include features and capabilities that pertain to admissions, billing and payments, reporting of student behavior and performance. In addition, SIS software generally includes apps or portals for parents, students, and third parties, thus making a SIS an effective tool for communication and consolidated data collection.

This manner in which data is formatted can vary widely, depending on the school. Accordingly, there can be a different import adapter 142 for each different student information system that a particular school distract might use. For example, larger school districts might use applications where the import adapter 142 has an available API integration, so the data is uploaded directly from the cloud 134, via its import adapter 142 component, into the SIS of the school (third party) 138. In the absence ofan available API integration, the import adapter 142 may make various data formats available to the school (third party) 138, such as: XML (eXtensible Markup Language), JSON (JavaScript Object Notation), and/or CSV (Computer System Validation)/Spreadsheet. Finally, the import adapter 142 can reproduce the existing paper forms so that they can be printed and scanned.

The import adapter 142 is based on a standard data model for the data extract from the vault 126, and this data model is independent of the mechanism required by the SIS for the import. This results in a pluggable “blade” architecture in which new “blades” can be quickly added to process new requested data formats or integrate with specific SIS applications.

A software blade is a logical building block that is independent, modular and centrally managed. Software blades can be quickly deployed and configured into a solution based on specific needs. As needs evolve, additional blades can be activated to extend functionality to an existing configuration within the same hardware foundation.

This idea of pluggable “blades” is also part of what makes gives the registry adapter 136 the ability to accommodate different registries while leveraging its common processing logic. Internally, the registry adapter 136 understands the same standard data format of the vault 126, but also understands an abstract model of how to forward a subscriber's 602 request for data to a SIR 114a-n, handle any exceptions and errors in the choreography with the SIR 114-an, and ultimately receive, process and understand the result.

Each time the system 100 establishes a new relationship with a SIR 114a-n, a new “blade” is added to the registry adapter 136 that encodes the data formats and process steps required. It is possible that the new “blade” may introduce new data items or processing steps to the core model supported by the registry adapter 136. In this case, the registry adapter's 136 core abstract model is extended to accommodate the new requirements. This approach can be thought of as an asymptotic process whereby these extensions become rarer and rarer as the core abstract model is generalized to handle more and more SIRs 114a-n.

Import adapter 142, in addition to interfacing and operating in the context of SIRs 114a-n, can also analogously operate in and be used in the context of an Emergency Medical Responder (EMR). That is, import adapter 142 can be similarly used in the context of EMRs to allow users 102 to allow their data stored in the vault 126, and accessed by authorized EMR personnel.

In addition, users 102 can also be given the option to indicate that they have opted out of a required vaccination for themselves or their minor child, for example, due to a religious or a medical exemption. The system 128 will then direct the user to the appropriate “Opt-Out” form(s) used by states for their current state (or jurisdiction) of residence.

As new vaccinations get added to SIRs 114a-n by, for example, healthcare providers 130, 132, those updates will also be added to the user's 102 information and stored in the vault 126. Other information, such as a user's genetic code can be added to and included in the user's 102 information and stored in vault 126. The addition of genetic codes would allow physicians to more accurately diagnose and prescribe therapies based on the member's individual genetic traits. The system 128 can transmit a notification to the user's 102 mobile phone 108, computer 106 or tablet 110 when, for example, new vaccination information or records has been stored in the vault 126. The SIRs 114a-n can, for example, be batch run daily to note any changes to user's 102 record(s) in a particular SIR 114a-n. User's 102 may also check and query one or more applicable SIRs 114a-n based, for example, on a member's residency history for updated records and information.

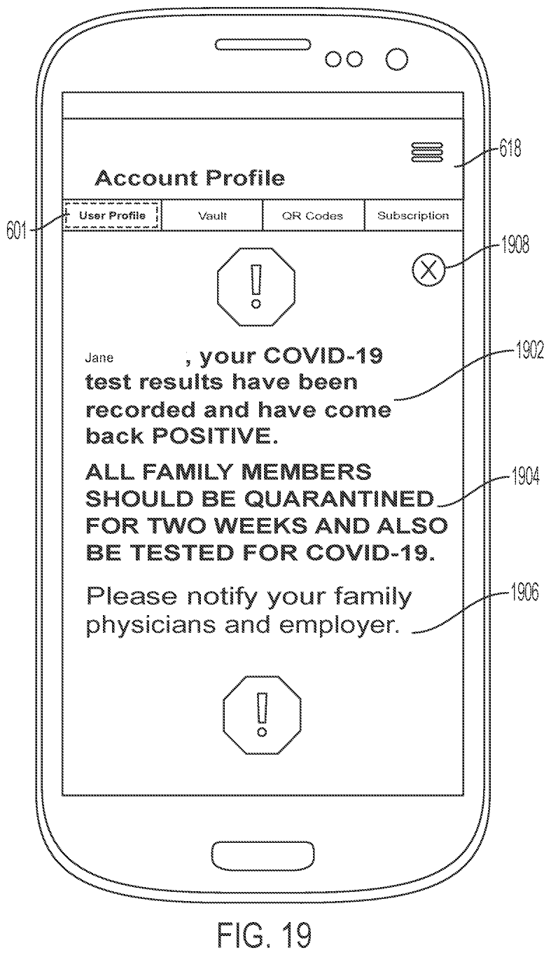

Vaccination record system 128 can receive the test results from a laboratory 140 or other medical facilities that has analyzed text specimens, in a same or similar manner as the system 128 receives records from SIRs 114a-n. For example, when record sharing service 120 receives test results from a laboratory 140, the results are stored and time stamped in the vault 126 record for the subscriber 602 or profile member 608a-e who has the test. The system 128 will then notify the subscriber 602 or profile member 608a-e that the test result has arrived, such as described below with regard to FIGS. 19-21.

In addition, healthcare providers 130, 132 can access SIRs 114a-n to find patient histories for new patients, and print reports for parents or guardians of minor children who need such records for school, childcare entry, and so forth. School nurses, for example, can use information stored in SIRs 114a-n to help find immunization records for immunization coverage reporting.

Because SIRs 114a-n generally contains records for all persons born in (or seeking medical care in) a particular state, healthcare providers 130, 132 are able to use it to access, revise and analyze explore immunization coverage in their own practice, and identify persons who are not up to date for immunizations. SIRs 114a-n can therefore be a valuable tool for assessing, for instance, pockets of need in the case of a disease outbreak, and for improving vaccination rates and reducing vaccine-preventable disease.

Generally, users 102 such as parents and guardians, and healthcare providers 130, 132 such as medical professionals and school nurses who administer immunizations or need access to immunization information for the purpose of delivering health care, may obtain access to the SIRs 114a-n by applying for an account with a particular state or jurisdiction.

The SIRs 114a-n are responsible for certifying that information is correct. A representative or other authorized personnel associated with each respective SIR 114a-n can electronically transmit the requested vaccination records in one or more SIRs 114a-n for storage in the vault 126. A mobile phone 108, a computer 106, or a tablet computer 110 of the user 102 can then access the vaccination records in the vault 126.

Although not expressly shown in FIG. 1, system 100 can also optionally utilize other known networking techniques such as the use of local area networks, the public switched telephone network (PSTN), the Internet, and the use of routers and firewalls, a virtual private network (VPN), and other encrypted or secure data transmission protocols are typically used in light of the sensitivity of user 102 data.

Embodiments of the invention also include search engines, automated decision-making modules, filters, user interfaces, and other computer-based technologies that can be implemented using various software components, such as modules, programs, scripts, and routines. Typically, these are resident on a vaccination record system 128. However, local versions can be configured on individual mobile phones 108, computers 106 and/or tablets 110.

FIG. 2 illustrates a block diagram of the internal hardware of the computer 106 of FIG. 1. Devices such as a mobile phone 108 and tablet computer 110 will generally be architecturally configured in a similar manner as computer 106.

A bus 214 serves as the main information highway interconnecting the other components of the computer system 200. CPU 216 is the central processing unit of the system, performing calculations and logic operations required to execute a program. Read only memory (ROM) 218 and random- access memory (RAM) 220 constitute the main memory of the computer.

Disk controller 222 interfaces one or more disk drives to the system bus 214. These disk drives are, for example, solid state drive 206, or internal or external hard drives (or disk) 224. Disk drive indications 224 and 206 are merely symbolic of a number of disk drives which might be accommodated by the computer 108. Vaccination record system 128 and immunization registries can also be utilized in connection with a computer 106, or a similar computing system.

Typically, computer 106 would include a hard disk drive 224 and a solid-state drive 206. The number and type of drives varies, typically with different computer configurations. Disk drives 224 and 206 are in fact optional, and for space considerations, can be omitted from the computer system 106 used in conjunction with the vaccination record system 128 described herein.

Camera controller 232 interfaces camera 234 to the system bus 214.

The computer 106 also has a display 208 upon which information is displayed. A display interface 226 interfaces display 208 and permits information from the bus 214 to be displayed on the display 208.

Communications with external devices, for example, the components of the apparatus described herein, occurs utilizing communication port 228. For example, optical fibers and/or electrical cables and/or conductors and/or optical communication (e.g., infrared, and the like) and/or wireless communication (e.g., radio frequency (RF), and the like) can be used as the transport medium between the external devices and communication port 228. Peripheral interface 230 interfaces the keyboard 210 and the mouse 212, permitting input data to be transmitted to the bus 214.

Infrared transmitters are optionally utilized when the computer 106 is used in conjunction with one or more of the processing components/stations that transmits/receives data via infrared signal transmission. Instead of utilizing an infrared transmitter or infrared receiver, the computer system optionally uses a low power radio transmitter and/or a low power radio receiver. The low power radio transmitter transmits the signal for reception by components of the production process and receives signals from the components via the low power radio receiver. The low power radio transmitter and/or receiver are standard devices in industry.

In some situations, a keyboard 210 and a mouse 212 are provided as input devices to interface with the central processing unit 202. Then again, for enhanced portability, keyboard 210 may be a limited function keyboard, or omitted in its entirety. In addition, mouse 212 optionally is a touch pad control device, or a track ball device, or even omitted in its entirety as well. In addition, the computer 106 also optionally includes at least one infrared transmitter and/or infrared received for either transmitting and/or receiving infrared signals, as described below.

Although computer 106 is illustrated having a single processor, a single hard disk drive and a single local memory, the system 106 is optionally suitably equipped with any multitude or combination of processors or storage devices. Computer 106 is, in point of fact, able to be replaced by, or combined with, any suitable processing system operative in accordance with the principles of the present invention, including sophisticated calculators, and hand-held, laptop/notebook, mini, mainframe and super computers, as well as processing system network combinations of the same.

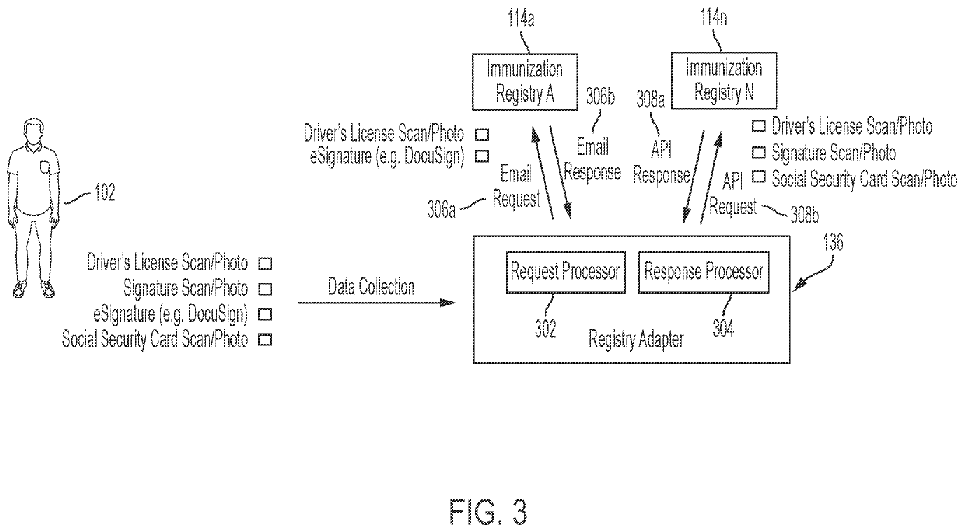

FIG. 3 shows a logical representation of the registry adapter 136 shown in FIG. 1. The registry adapter 136 is part of the record retrieval service 118, and can run as a service in AWS, but is not an AWS developed service.

The registry adapter 136 emulates the process that would be followed by a user 102 using the existing request process as defined by, for example, a particular SIR 114a-n.

The registry adapter 136 encapsulates, for example, the differences in process, technology, data requirements, and data formatting that pertain to individual SIRs 114a-n into a single component. So any SIR 114a-n (or other registry) will be interacting with the channel in a particular SIR (e.g., SIR 114a) that is specifically developed for that SIR (e.g., SIR 114a).

As shown in FIG. 1, the retrieval service 118 interfaces with the registry adapter 136 through a common service interface. For example, the retrieval service 118 might start servicing a user 102 request to obtain records from the vault 126 that are maintained by a particular (e.g. Georgia), so it would ask the registry adapter “what data is required for Georgia”, and it would receive an answer like “driver's license and birth certificate”, which it would collect from the user 102.

The retrieval service 118 would call the registry adapter 136 to submit copies of these documents for the user 102 to the SIR (e.g., SIR 114a) for the state of Georgia (A to SIR XY″). The registry adapter 136 would know how to format the required form, fill in the details, compose an email, attach the documents correctly, and send the request, formatting the email so the reply is returned back to the registry adapter 136 from the SIR 114a in this case.

When the registry adapter 136 receives the response, it understands the reply email sufficiently to call back to the retrieval service 118 indicating that the requested record for the user 102 has been received from SIR 114a, in this case.

In an embodiment, an accepted Immunization Information Systems (IIS) Health Level 7 (HL7) specification, such as version 2.5.1, which is incorporated herein by reference in its entirety, can be used to provide automated feeds to SIRs 114a-n that utilize the specification. SIRs 114a-n may also, for example, accept HL7 specification 2.5.1 files in batch uploads. For example, for the state of Georgia Department of Health, the user 102 needs to complete a “Request for State of Georgia Official Immunization Record,” which is available at dph.georgia.gov, and states “all immunization record requests must be accompanied by documents that identify the person requesting the immunization record. Examples of acceptable forms of identification: a state-issued photo driver's license with address, a state-issued photo identification card with address or a U.S. passport or passport card with photo. Please lighten the copy of the identification cards. If the record requested is for a minor under 18 years of age, please state your relationship to the minor in the ‘Requestor's Relationship’ field.”

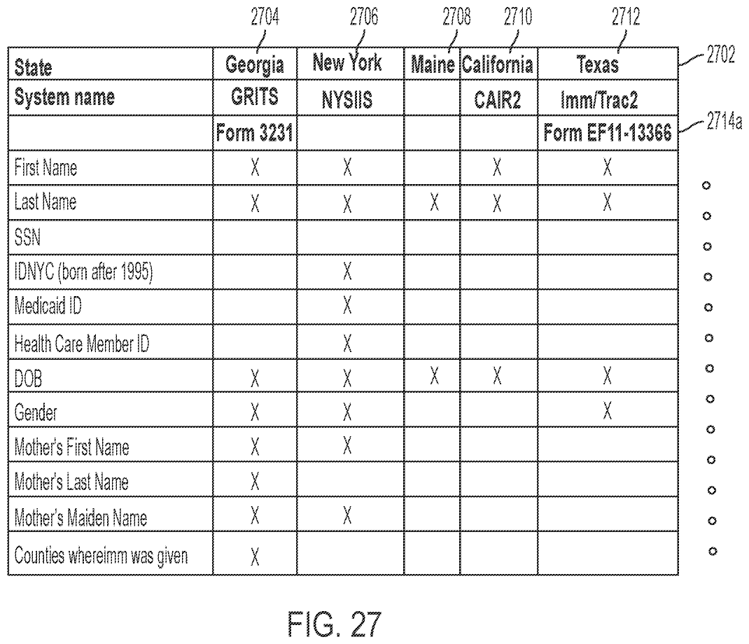

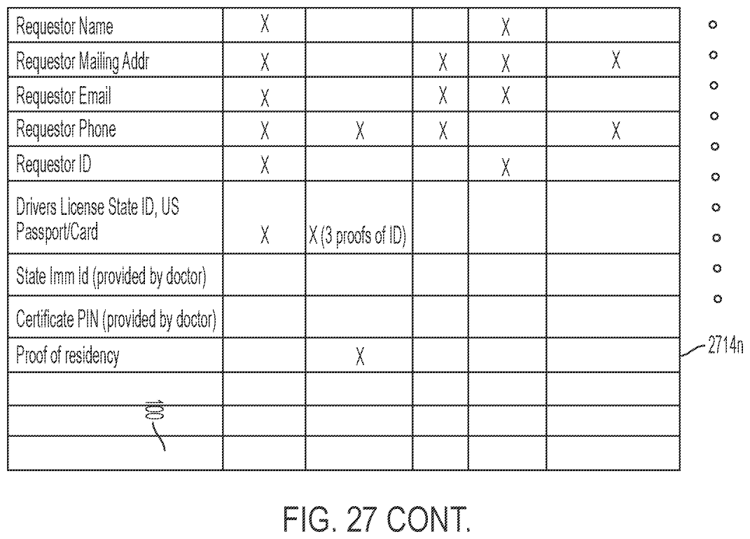

Registry adapter 136 would collect the data required from the user registry 124 that is needed to complete the forms that the registry adapter 138 knows about. The data can include, for example, the user's 102 name, date of birth, counties where vaccinated, gender, mother's name, address, and email address, such as shown in FIG. 27 at column 2704 for the state of Georgia. The registry adapter 136 will then format this data into a .pdf format (as requested by a particular SIR 114a-n), collect one or more accompanying documents 310, 312, 314, 316 that may be required by a particular SIR 114a-n.

For the state of Georgia, as described above, a state-issued photo ID, a driver's license or a passport 310 can be used. The registry adapter 136 will also, for example, format a signature in a form acceptable to the SIR114a-n, using an approved eSignature, such as DocuSign. Registry adapter 136 will also assemble all of the above information that is required by the “Request for State of Georgia Official Immunization Record,” which is available at dph.georgia.gov, into an email transmission to the required email address, such as dph-immreg(ii)dp1u;rn.gov for the state of Georgia. The registry adapter 136 can use an email sender and/or reply-to format that routes the response back to the registry adapter 136, which is part of the record retrieval service 118.

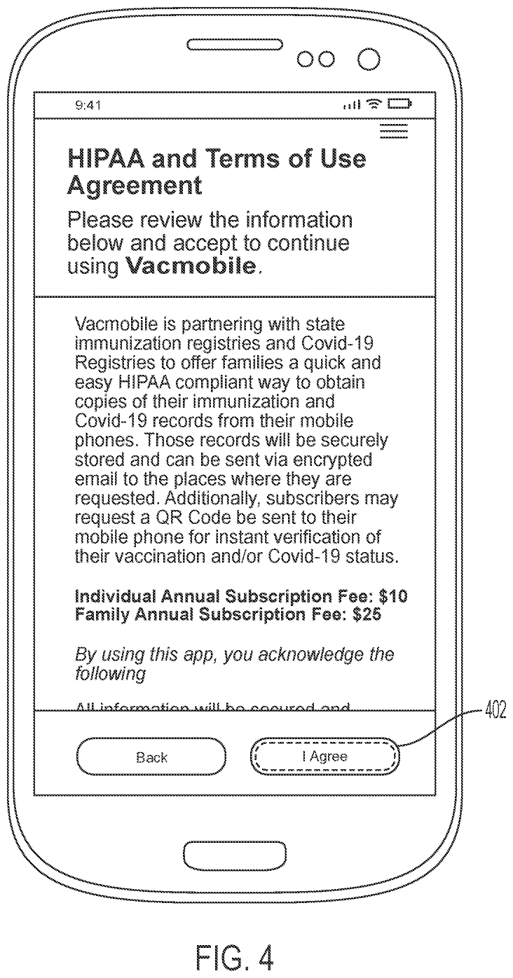

An embodiment of registry adapter 136 can include a request function 302 and a response function 304, which can optionally be combined into a single functional module if desired. In a more general sense, the registry adapter 136 knows the information required to issue a request for each SIR 114a-n. In addition, the registry adapter 136 knows the format of the response it received from each SIR 114a-n. In addition, registry adapter 136 knows the technology and process steps to interact with each SIR 114a-n, and can request a single set of data elements from a user that aggregates the information needed by multiple SIRs 114a-n. FIG. 4 is a screen shot of a HIPAA and Terms of User interface, for use with the system of FIG. 1. After selecting icon 402, the user 102 can begin setting up the account by being presented with one or more screens (not shown) that logically interface and interact with enrollment service 116 shown in FIG. 1 to allow a user 102 to enter profile information such as: first name, middle name, last name (and suffix, if applicable), maiden name, additional names, email address, and password.

FIG. 27 shows such exemplary user profile information. As shown in the row labeled “State”, exemplary states of Georgia 2704, New York 2706, Maine 2708, California 2710 and Texas 2712 are shown. Additional states, United States territories, Nations, Tribes and Pueblos and other country jurisdictions can, and generally will be included. Generally, each entity can utilize or include varying and different profile information as provided in rows 2714a-n. Such information for each entity can be added, deleted and/or modified over time.

As part of setting up an account, the user 102 will also be presented with one or more screens (not shown) that allow a user 102 to enter profile information such as the name of immunizations received, and the county and state and/or originating entity where each immunization was received. Finally, when setting up an account, the user 102 can enter payment information such as by credit card, PayPal, Apple Pay, Google Pay, etc.

Payments can be, for example, monthly or yearly, for an individual or for a family.

When a user 102 logs into their account, two-factor authentication will add a layer of security. For example, when a user 102 logs into their account, the user's 102 password is entered. The user 102 would then receive, for example, a code via text on the mobile phone 108 that would also need to be entered to log in to the account. The second layer in two-factor authentication means that an unauthorized user would need to obtain the user's 102 password as well as the user's mobile phone 108 to access the user's 102 account and information stored in the vault 126. Additionally, the device (mobile phone 108) used to access the vault will be verified if that device has not previously been used to access the member's account.

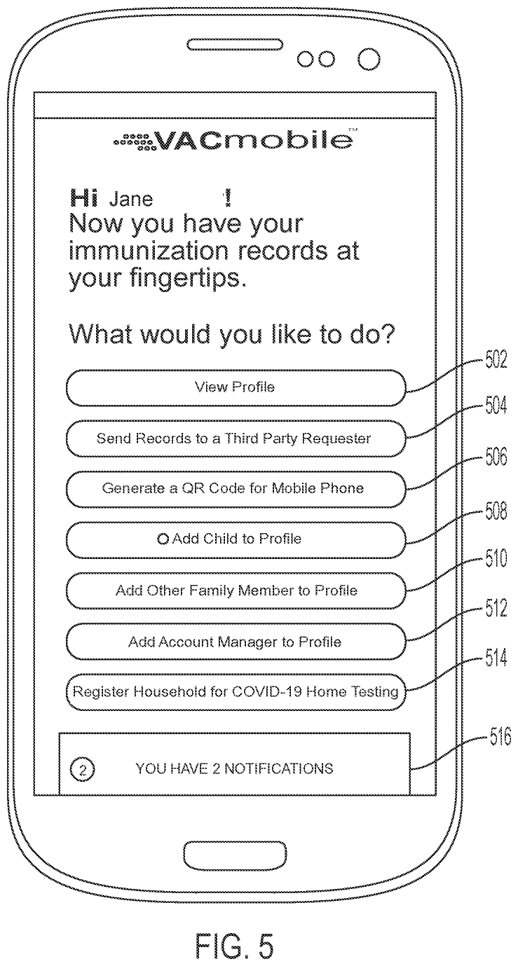

FIG. 5 is a screen shot showing a user home page. Icons 502, 504, 506, 508, 510, 512, and 514 allow the user 102 to initiate various actions, as described herein. For example, profile information for children can be added by selecting icon 508, and one or more screen displays will be provided to enter the profile name and immunization information of a child. In addition, additional adult family members can be added by selecting icon 510, and one or more screen displays will be provided to enter the name and immunization information of such adult family member(s). A user can select icon 516 to read new notifications or messages. The system 100 can use end-to-end encryption to facilitate secure transmission of user 102 information such as vaccination and COVID-19 status health records when icon 504 is selected.

FIG. 6 is a screen shot showing an account profile 618. In an embodiment, an account profile can include a user profile tab 601, a vault tab 610, a QR codes tab, and a subscription 620 tab. When user profile tab 601 is selected, in an embodiment, the subscriber name 602 that originated the account profile, as described above with regard to FIG. 4, is displayed when user profile tab 601 is selected.

The user profile 600 of any user, such as subscriber name 602 or profile members 608a-e, that is added and included under the user profile tab 601 can include information that is the same as or similar to at least a portion of the profile information as provided in rows 2714a-n of FIG. 27. For example, a user profile 600 can include information such as the user 102 (subscriber) name 602, login and security information 604, contact information 606, and profile members 608. Profile members 608a-e may be added by pressing icon 512 and or 514 to provide exemplary information. Specifically, profile members 610a and 610b are added by selecting icon 510 in FIG. 5, and profile members 608c-e are added by selecting icon 508 in FIG. 5. Messages 614,616 correspond to the two notifications indicated by icon 516 in FIG. 5.

Generally, a user 102 of the system 100 can be the subscriber name 602 or any of one or more of profile members 608a-e. However, in an embodiment, only the subscriber name 602 or profile members 608a-e that have the role of account manager 620 have authorization to perform functions such as adding a child 518, adding another family member 510, and changing or updating payment information. An account manager 620 can be thought of someone that would might take the subscriber 602 to a medical emergency room in an urgent situation, and therefore may need to access or find it beneficial to the subscriber's 602 record in the vault 602 in order, for example, to share that information with a first responder or with health care professionals on behalf of the subscriber 602 if the subscriber 602 was incapacitated. An account manager 618 is also someone that can make sure that the subscriber 602 adheres to the recommended vaccinations as the push notifications come in.

For example, an account manager 618 would receive and see notifications and messages in the account manager's 618 record in the vault 602 that the subscriber 602 receives and that profile members 608a-e receive, in the same manner that subscriber 602 receives and that profile members 608a-e receive such messages and notifications. In an embodiment, the system 100, vaccination record system 128 or components thereof include security to ensure that a subscriber's 602 vault 126 record or a profile member's 608a-e vault 126 can only be accessed by the respective subscriber 602, profile member's 608a-e, or the account manager 618 can access the respective records stored in the vault 126. In another embodiment, a subscriber's 602 vault 126 record or a profile member's 608a-e vault 126 record can be accessed by, for example, first responders and/or emergency healthcare providers who can provide a medical alert override to obtain access to a subscriber's 602 vault 126 record or a profile member's 608a-e vault 126 record. The account manager 618 role can be useful, for example, for people who are caretakers for an aging parent, a disabled family member or relative, and the like.

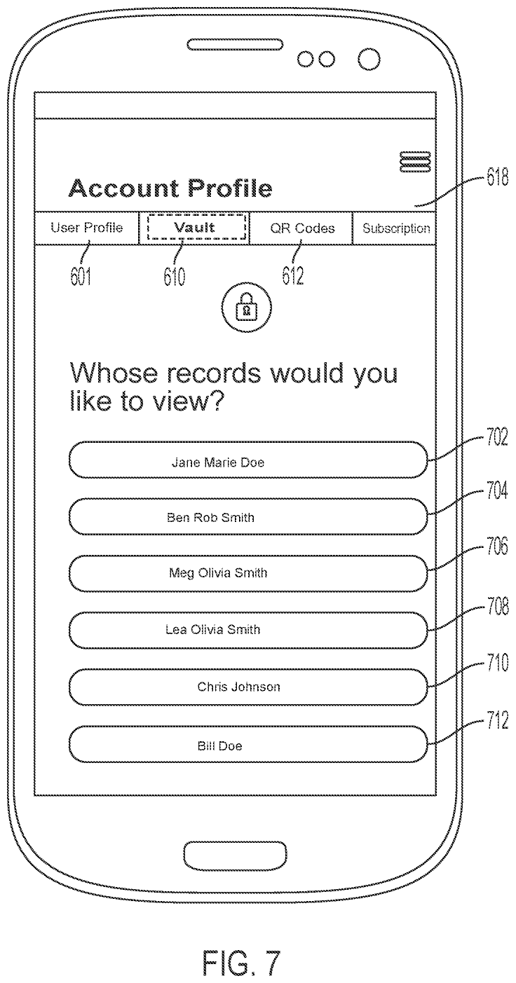

FIG. 7 is a screen shot that is shown when the vault tab 610 is selected in FIG. 6. The subscriber name 602 corresponds with icon 702. The profile member 608a-e names that respectively appear within icons 704, 706, 708, 710 and 712 have been added by selecting, as appropriate, icon 508 and 510 to provide exemplary information. The records associated with the names that respectively appear within icons 704, 706, 708, 710 and 712 are stored in the vault 126 of FIG. 1.

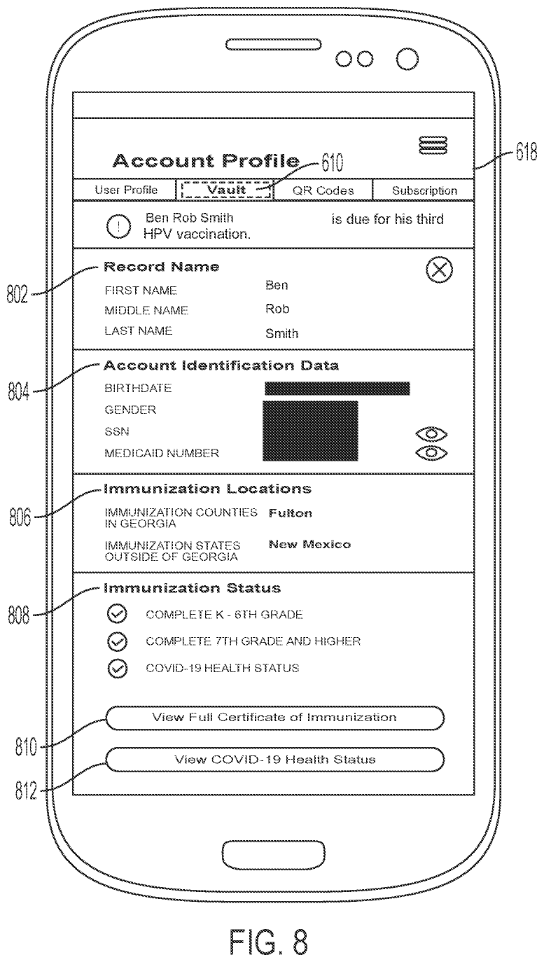

FIG. 8 is a screen shot for profile member 608c that appears under vault tab 610 when icon 704 is selected. Enrollment service 116 shown in FIG. 1 allows a subscriber name 602 or account manager 620 to input information such as record name 802, account identification data 804 for the record name 802, and immunization locations 806 for the record name 802. Upon entering the record name 802, account identification data 804 and immunization locations 806, the system 100 will check SIRs 114a-n as appropriate and provide an indication of immunization status 808.

As shown in FIG. 8, the immunization status 808 indicates that immunizations are complete for 6th grade, 7th grade and higher, and for COVID-19 . Alternatively, if, upon checking one or more appropriate SIRs 114a-n, a particular state registry indicates that immunization status 808 is not up-to-date or complete, an indication of any immunization that are recommended or required can be identified and displayed. Icon 810 allows a full certificate of immunization to be viewed, and icon 812 allows COVID-19 health status information to be viewed.

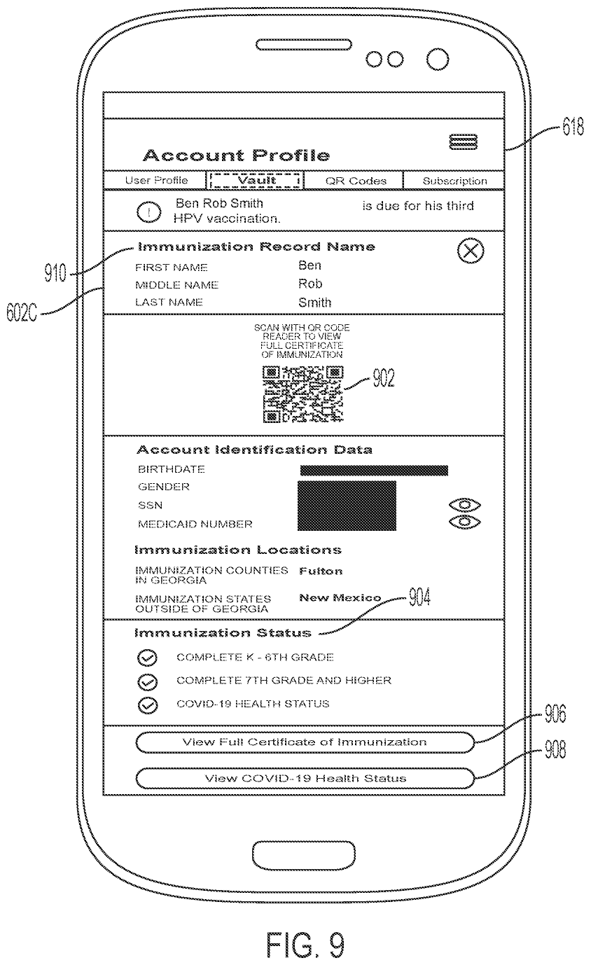

FIG. 9 is a screen shot that appears when View Full Certificate of Immunization icon 810 is selected in FIG. 8. The immunization record 910 is shown for profile member 608c. A full certificate of immunization can be obtained by reading QR code 902 or by selecting icon 906. Immunization status is also provided at 904 (and also shown at 808). While QR code is used for exemplary purposes, it is understood that any scannable identifier can be used in addition to or in place of a QR code.

The option to obtain COVID-19 health status can be obtained by selecting icon 908. QR code 902 can also be used by a third party 138, such as, for example, i) a school or daycare center, that the subscriber 602 or a profile member 602a-e attends, ii) the employer of the subscriber 602 or a profile member 602a-e, or iii) a business, public transit, restaurant or hospitality industry location that the subscriber 602 or a profile member 602a-e is visiting. The third party 138 can scan the QR code 902.

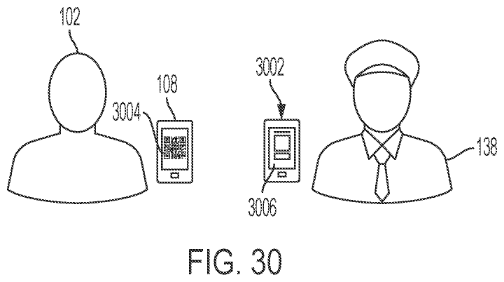

Scanning the QR code with a QR code reader (such as shown in FIGS. 29 and 30) will typically take the third party 138 to the user's immunization record stored in vault 126, as current QR codes are limited to less than 3k of binary data. For example, a URL (Uniform Resource Locator) can be encoded in the QR code that would direct the third party 138 to the user's immunization record or COVID-19 health status stored in vault 126. For example, a QR code that includes a URL that leads to a .pdf file can be generated in a conventional manner. The subscriber's 602 or profile member's 602a-e immunization information can be stored in vault 126 by using, for example, a conventional relational database program, or in .pdf format. The use of QR codes involves a single subscriber 602 at a time, generally in the context of a point-in-time interactive use case, such as admission to an arena or a first responder providing care.

After scanning the QR code, access to the subscriber's 602 or profile member's 602a-e immunization information can be further restricted. For example, in .pdf format, a password option can be utilized so that the user 102 can enter a password that would need to be entered to open the .pdf file containing the user's vaccination information. The system can provide a document (e.g., a token, a PDF file, a file, a word processor file, a data structure, an array of data, etc.) to the third party 138. The document can include a digital certificate signed by a trusted certificate authority. The system 100 can provide a list of trusted authorities that are allowed to sign certificates. The document can be digitally signed by a private key of the trusted authority such that a public key can verify the signature but not generate the signature. The system 100 can determine if the digital certificate is valid, has not been revoked, and is within a valid time window (e.g., not expired). In some embodiments, the third party 138 can be required to download a secure application that executes on a computing device of the third party 138 to show various data in the document (e.g., a health pass, a vaccination record, or other information as discussed herein). The secure application can ensure the third party 138 is authorized to access the document.

A password for the document can be also sent to the third party 138 in any desired manner, so that the intended recipient can enter the password and open the document after scanning the QR code the leads to the document that includes the immunization record. As one example, a PDF document can advantageously be updated with new vaccination records and information and accessed with the original QR code and password. In some embodiments, the document can be transmitted to the secure application in an encrypted format. The secure application can generate a secure storage on the computing device of the third party 138 by generating or accessing encryption keys to store the information in an encrypted format. In some embodiments, the secure application can request access to the locally stored file from the vaccination record system 128. In some embodiments, the secured application can only grant access to the third party 138 upon receiving confirmation from the vaccination record system 128. In one embodiment, the vaccination record system 128 can transmit a time-sensitive token to confirm that the document can be opened and displayed. The vaccination record system 128 can generate a log of each access for audit purposes, such as, for example, in a blockchain ledger or data store.

The system 100 can allow users the ability to revoke access by one or more third parties 138 at any time such that future attempts to access the patient's data are denied. In some embodiments, the system 100 can generate a request to a computing device of the user 102 (e.g., mobile phone 108, desktop or laptop computer 106, or tablet computer 110, etc.) to authorize each access of a user's data by a third party 138. The system 100 can receive a confirmation or rejection from the user, and inform the secure application whether to provide access to the user's data.

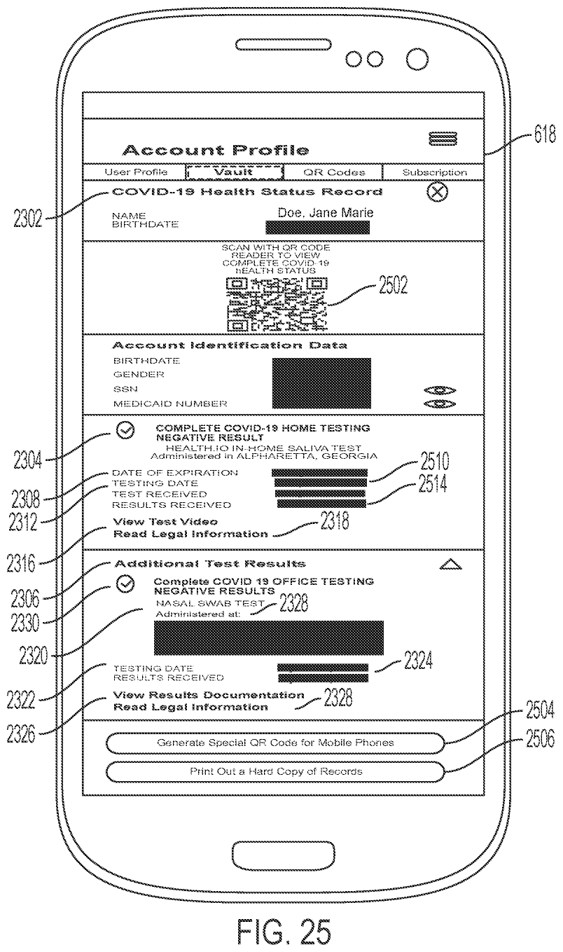

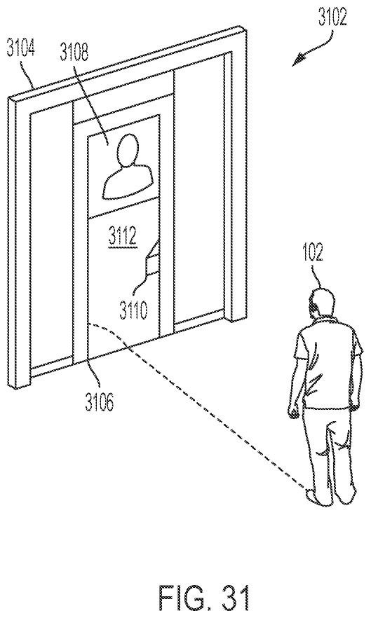

The QR code reader (such as shown in FIGS. 30 and 31) may utilize, for example, open source software or propriety software, or a combination thereof. The QR code reader may be, for example, an app that reads the QR code such as shown in FIGS. 9 and 25. As discussed above, a third party 138 can scan QR code 902 with an electronic device of the third party 138 (such as device similar to the mobile phone 108, computer 106 or tablet 110 shown in FIG. 1).

FIG. 30 shows the use of a device 3002 that has a companion app 3006 that is used by a third party 138 to read a subscriber's QR code 3004 such as generated in FIGS. 9 and/or 25, and/or a subscriber's biometric data to obtain access to the subscriber's immunization records and/or COVID-19 status. The companion app 3006 on device 3002 can also be used to read biometric data of the user 102, such the use's facial data for facial recognition identification, iris scanning and/or fingerprint data. The companion app 3006 and/or the vaccination record system 128 can authenticate the biometric information to authorize the user to access. Upon authenticating the user 102 by, for example, comparing the scanned data with the user's 102 biometric data stored in the user registry 124, the device 2902 can obtain access to the user's 102 immunization records and/or COVID-19 status, which can be presented on a display screen of the device 3002.

FIG. 31 shows the use of a device 3102 that has a companion app 3110 that is used read a user's QR code such as generated in FIGS. 9 and/or 25. The companion app 3110 can reside on a device 3002 such as shown in FIG.

FIG. 30. The device 3102 also includes an outer frame 3104, a passageway frame 3106, and a passageway 3112 for the user 102 to walk through. Biometric reader 3108 can also be used to read biometric data of the user 102, such the use's facial data for facial recognition identification, iris scanning and/or fingerprint data. Upon reading confirming such biometric data of the user 102 by, for example, comparing the read biometric data with the user's 102 biometric data stored in the user registry 124, the device 3002 can obtain access to the user's 102 immunization records and/or COVID-19 status. If the user's 102 immunization records and/or COVID-19 status is up to date and or in compliance with desired standards or requirements, the user 102 is allows to pass through the passageway 3012.

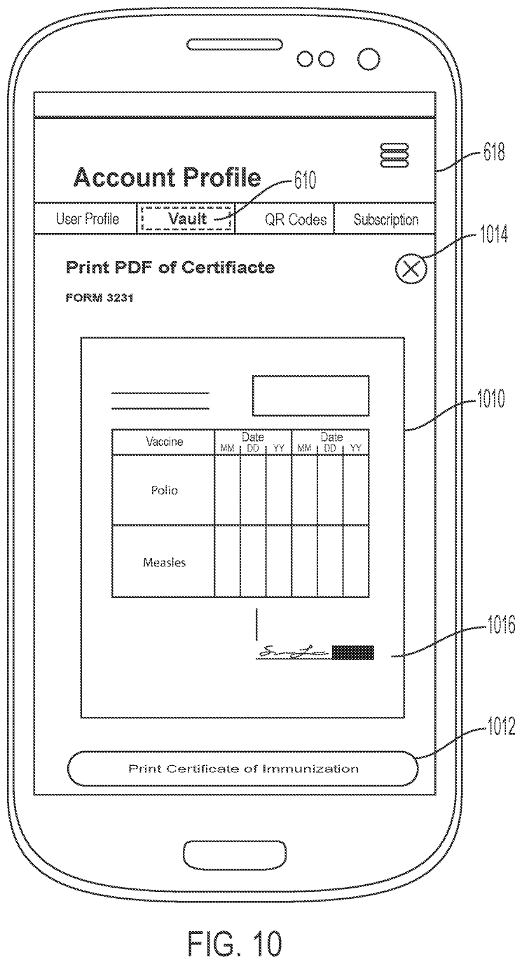

FIG. 10 is a screen shot showing a full certificate of immunization 1010 when either QR code 902 is scanned or when icon 906 is selected in FIG.9. A full certificate of immunization 1010 can be printed by selecting icon 1012. For example, a Certificate of Immunization (Form 3231) from the Georgia Department of Public Health can be displayed, printed, and/or transmitted to one or more third parties 138. Similar or analogous certificates of immunization from other states or jurisdictions can also be displayed, printed, and/or transmitted to one or more third parties 138.

The certificate of immunization 1010 is a digital version of the record that is stored in SIRs 114a-n, is therefore what is required by law in whatever state or jurisdiction the subscriber 602 or profile member 608a-e, along with an authorized signature 1016 of a duly authorized state (or jurisdiction) employee who is certifying the information from a particular SIR 114a-n. After either the full certificate of immunization 1010 is printed by selected icon 1012, or icon 1014 is selected if printing is not desired, the subscriber 602 is returned to the screen shot as shown in FIG. 5.

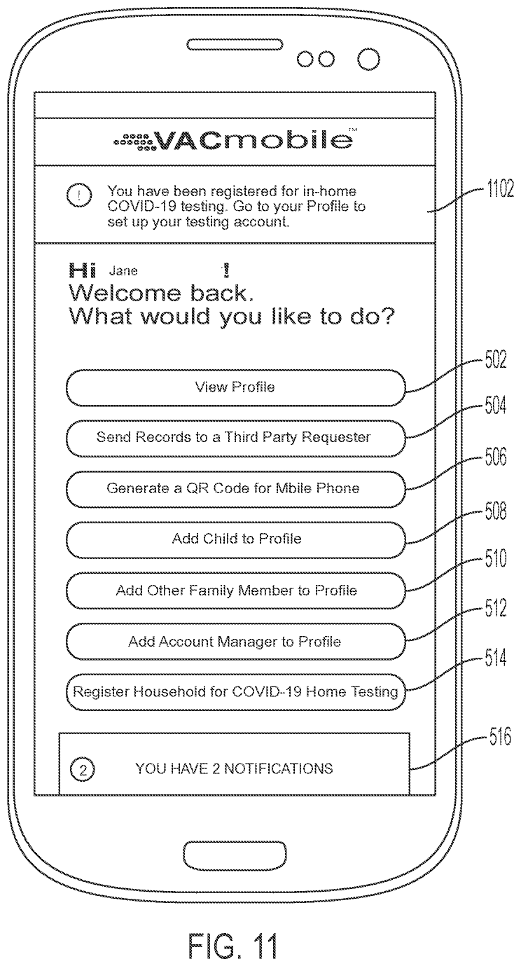

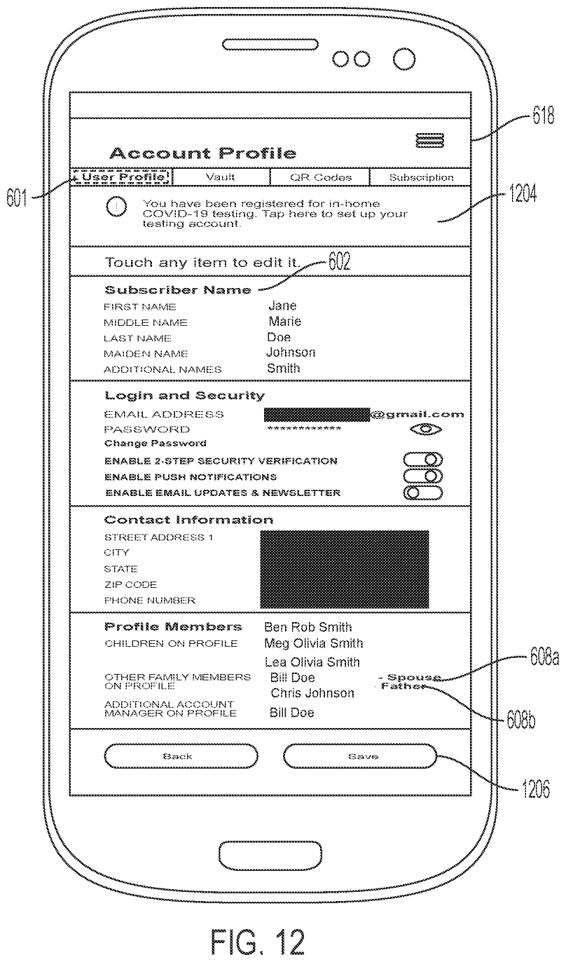

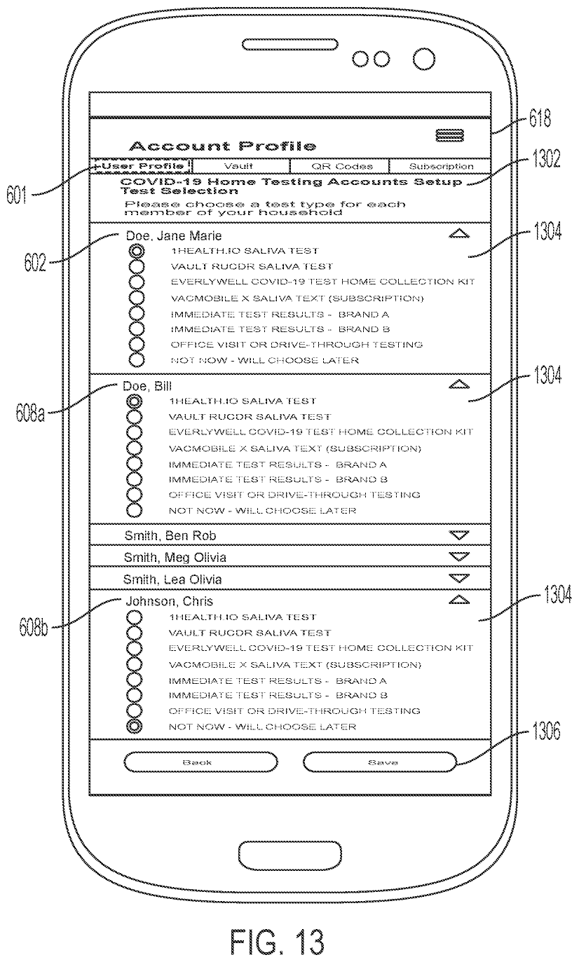

FIG. 11 is a screen shot similar to that of FIG. 5. The screen shot will be displayed when the subscriber 602 or profile member 602a-e is registered for COVID-19 testing by selecting icon 514 in FIG. 5. Registration is described with regard to FIG. 13 below. As shown in FIG. 11, after registration, message 1102 appears and states that the subscriber 602 has been registered for in-home COVID-19 testing. FIG. 12 is a screen shot indicating that subscriber 602 has been registered for COVID-19 testing, as indicated by the information displayed under the user profile tab 601 and by message 1204.

When the register household for COVID-19 home testing icon 514 in FIG. 5 is selected, the subscriber 602 is taken to the screen shot as shown in FIG. 13. FIG. 13 can be preceded, for example, by a conventional drop-down menu (not shown) that includes that a list of the names of subscriber 602 and profile members 608a-e that can be selected to participate in COVID-19 testing, and an acknowledgement form indicating that any selected profile member(s) 608a-e or subscriber 602 can physically or electronically sign to indicate that they consent to COVID-19 testing. After the COVID-19 subscriber name 602, for example, is selected from the dropdown menu, the subscriber associated with the subscriber name 602 selects the save 1206 icon, and a screen shot as shown in FIG. 13 appears.

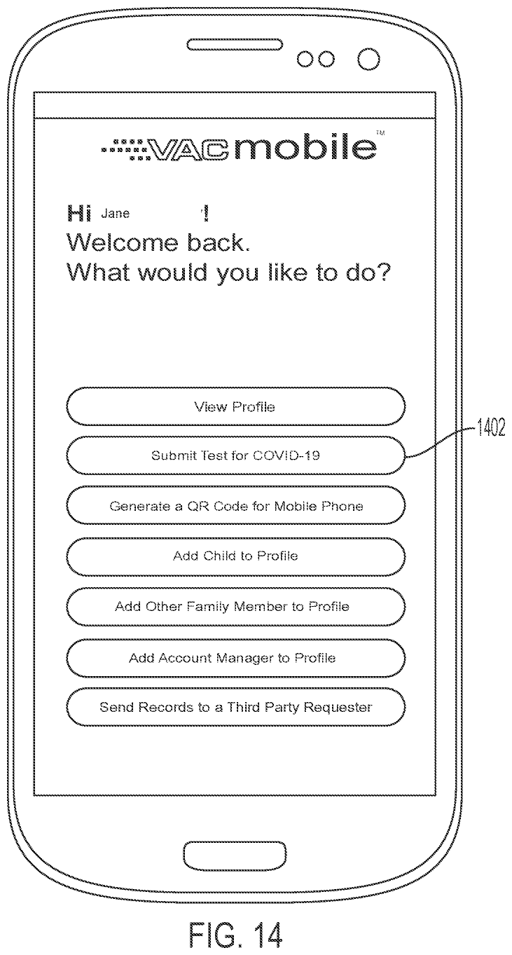

FIG. 13 is a screen shot that allows the subscriber 602 and/or profile members 608 to select a COVID-19 test, as indicated by message 1302. FIG. 13 indicates that subscriber 602 has selected a COVID-19 test from menu 1304, profile member 608a has selected a COVID-19 test from menu 1304, and that profile member 608b may later choose COVID-19 testing from menu 1304. Generally, menu 1304 the subscriber 602 and profile members 608a-e to any known method of testing. For example, for a test that provides results without having to mail in the test specimen to a testing laboratory 140 or other medical facility for analysis, the video 1614 can be used to verify and confirm that the subscriber 602 or profile member 608a-e is not swapping out the test strip with another test strip in which a color indicator on the strip corresponds to a test result. For example, a red color can correspond to a positive test result, and a green color can correspond to a negative test result. After the save icon 1306 is selected, the screen shot of FIG. 14 is presented.

FIG. 14 is a screen shot that allows COVID-19 enrolled subscriber 602 or profile members 608a-e to commence an embodiment of the COVID-19 testing process and COVID-19 test submission, by selecting icon 1402.

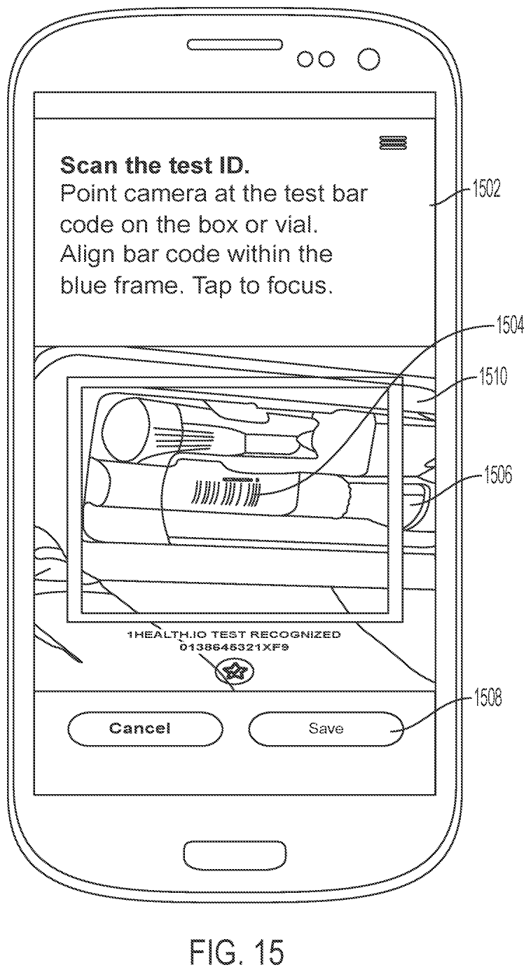

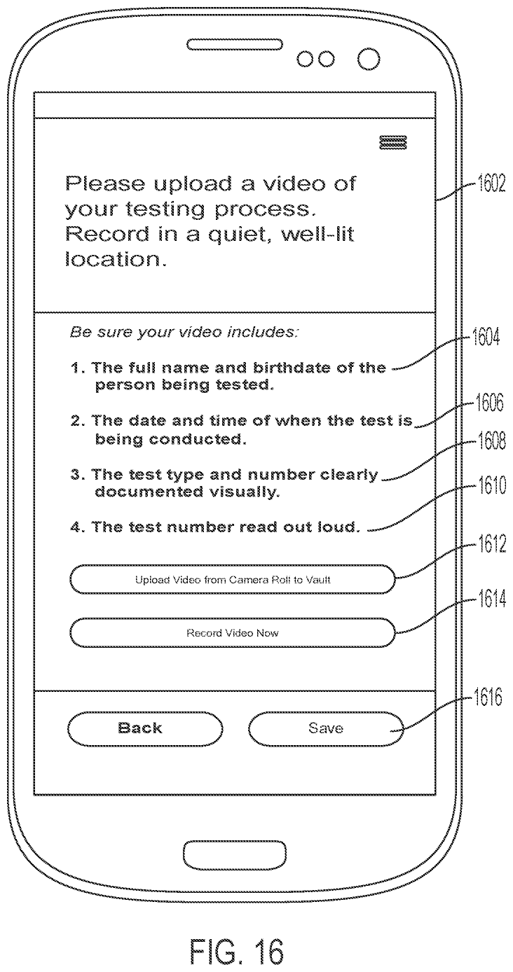

Upon selecting icon 1402, the screen shot of FIG. 15 appears, which commences the COVID-19 testing process. An instruction message 1502 that provides general instructions for the COVID-19 testing process can appear at the top of the screen. A conventional camera (not shown), such as on a mobile phone 108, can be used to take a photograph of the bar code 1504 associated with a COVID-19 test kit 1506. Upon completing the test within the test package 1510, the subscriber can mail the test specimen, for example, to a testing laboratory 140 or other medical facility 140 for analysis. Upon selecting the Save icon 1508, the screen shot of FIG. 16 is presented.

FIG. 16 is a screen shot that allows the uploading of a video of the COVID-19 testing process. An instruction message 1602 that provides general instructions for uploading a video that memorializes the COVID-19 testing process can appear at the top of the screen. More specific instructions that pertain to information preferably included in the video can relate, for example, to: i) the full name and birthdate of the person being tested (1604); ii) the date and time of when the test is being conducted (1606); iii) the test type and number documented visually (1608); and iv) the test number read out loud (1610). The subscriber can commence the recording by selecting icon 1614.

Additional information, such as the location of where the COVID-19 test is being conducted, can also be optionally included.

Upon completion of the recorded video, icon 1614 can be selected. Upon completion of the video recording in accordance with instructions 1602-1610, icon 1612 can be selected to facilitate the uploading of the video to the subscriber's record in the vault 126. Finally, after uploading the video pursuant to the selection of icon 1612, icon 1616 can be selected to save the recorded video and associate the recorded video with the information included in the subscriber's user profile tab 601 and record in the vault 126.

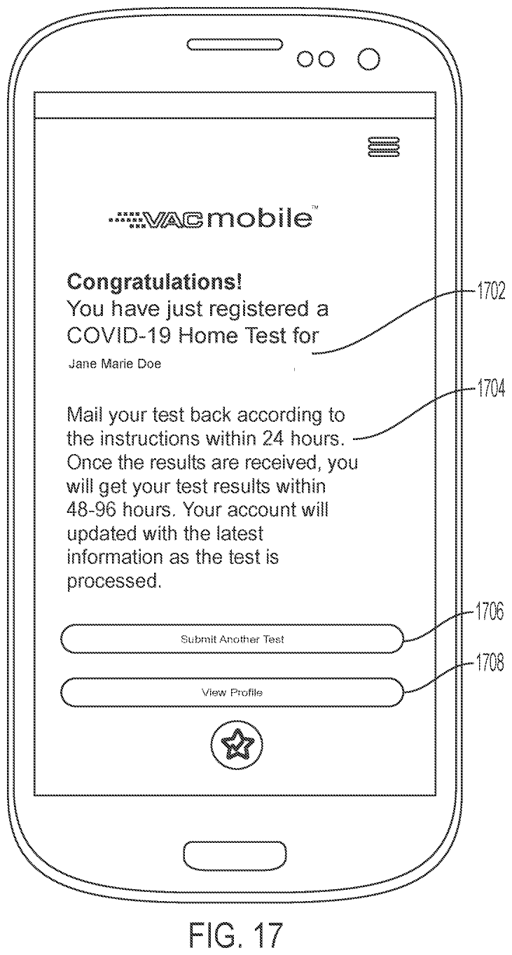

Icons are provided to indicate that video recording will commence (1614). After the video is recorded, Save icon 1616 can be selected to memorialize that recorded video has been uploaded to the system 128 and stored in the vault 126. Upon saving the test, the screen shot of FIG. 17 is presented.

FIG. 17 is a screen shot indicating that the COVID-19 test of FIG. 16 has been registered 1702 with a COVID-19 testing facility 140. Instructions are provided to mail the test to the laboratory 140 or other medical facility 140 that will conduct the analysis of the subscriber's 602 test package 1510, within a specified time period, such as 24 hours (1704). The testing laboratory 140 or other medical facility that analyzes the subscriber's 602 test sample will also provide the test results to the system 128 so that the results of the analysis of the subscriber's 602 test package 1510 can be stored in the vault 126 for the subscriber 602 (or profile member 608) that was tested. For SIRs 114a-n that include (or validate) COVID-19 testing results or information, such COVID-19 testing results or information can be transmitted from the SIRs 114a-n to vault 126 is a same or similar manner as described above with regard to FIGS. 1 and 3. Accordingly, as new COVID-19 testing results or information get added to SIRs 114a-n by, for example, healthcare providers 130, 132 and/or a testing lab 140, those updates will also be added to the subscriber's 102 or profile member's 608a-e information and stored in the vault 126 record, for that particular subscriber 602 or profile member 608a-e.

If icon 1706 is selected, the screen shot of FIG. 14 will be presented so the subscriber can submit another test for the subscriber 602 or a profile member 608a-e by selecting icon 1402. If icon 1708 is selected, the screen shot of FIG. 6 can be presented for the subscriber 602. Alternatively, a profile for another profile member 608a-e can be selected via a screen shot similar to that of FIG. 7.

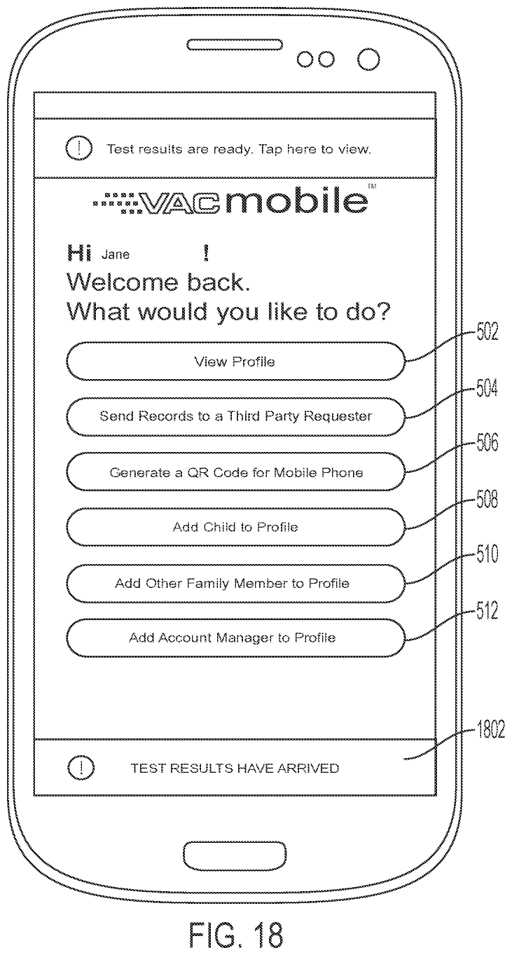

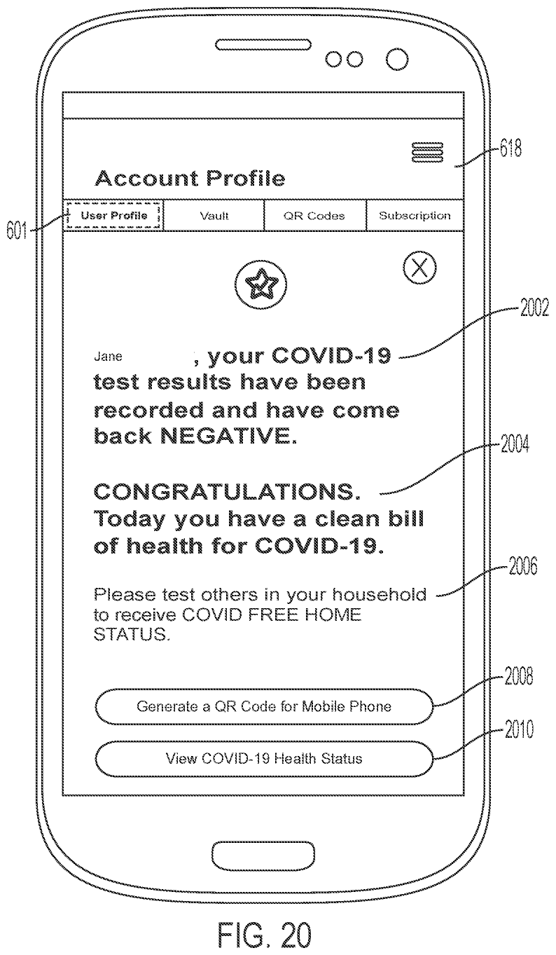

FIG. 18 is a screen shot of a subscriber name 602 similar to that of FIG. 5. In FIG. 18, icon 514 is not present, as the household for subscriber 602 has already been registered. In addition, FIG. 18 includes a message 1802 indicating that the COVID-19 test results have arrived from the healthcare provider that conducted the analysis of the subscriber's 602 test package 1510. Upon seeing the message 1802, subscriber 602 can either select the view profile icon 502 or the message 1802, either of which will then display the screen shot as shown in either FIG. 19 or FIG. 20, depending on the outcome of the COVID-19 test result, as described below.

If the subscriber's 602 COVID-19 test results have come back positive, the screen shot of FIG. 19 will be displayed. FIG. 19 includes a message 1902 indicating that the subscriber's 602 COVID-19 test results have come back positive, along with additional instructions 1904, 1906. After reviewing the message 1902 and information 1904, 1906, the icon 1908 can be selected to return, for example, to the screen shot of FIG. 6.

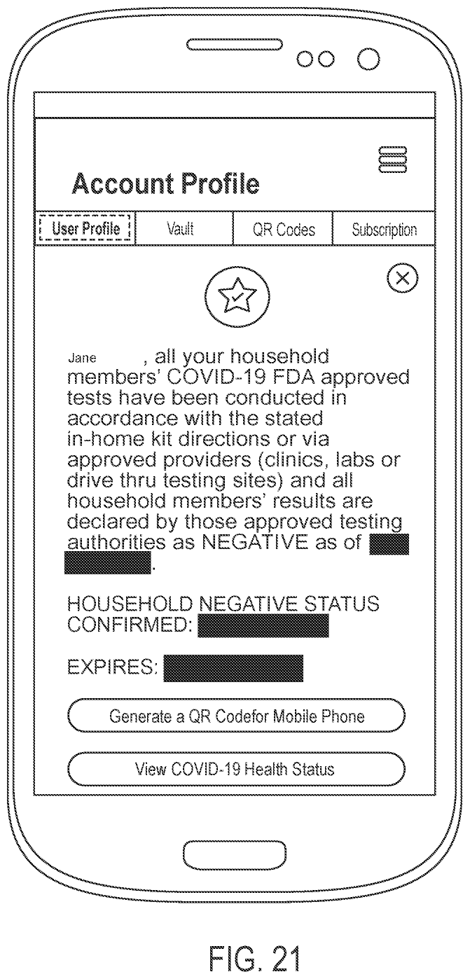

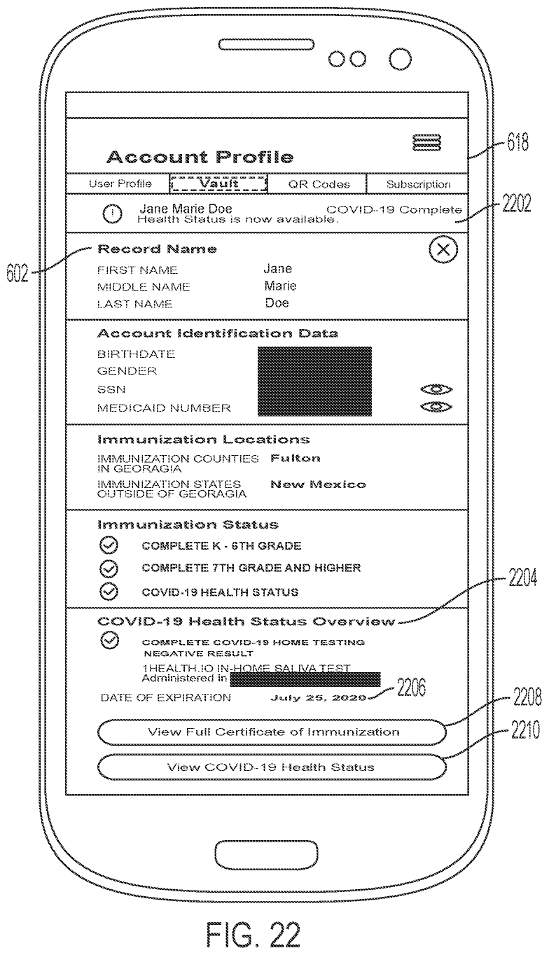

If the subscriber's 602 COVID-19 test results have come back negative, the screen shot of FIG. 20 will be displayed. FIG. 20 includes a message 2002 indicating that the subscriber's 602 COVID-19 test results have come back negative. One or more additional messages 2004, 2006 may also be displayed. Upon receiving the negative test result, icon 2008 can be selected to generate a new QR code, such as described above with regard to FIG. 9, that includes, for example, the negative test result and date of the negative test result. If icon 2010 is selected, the screen shot of either FIG. 21 or FIG. 22 can be displayed, as described below.

Generally, there are specific criteria that must be satisfied or performed for a Certificate of Immunization from any state or jurisdiction to be valid. Such criteria can vary for different states and jurisdictions.

For example, some states may merge immunization records for an individual into an overall “Health Certificate,” which includes, for example, a summary of the child's annual physical. A Certificate of Immunization, Health Certificate, or similar official document will have specific items to be present, such as a legible signature 1016, for example, of a duly authorized state (or jurisdiction) employee who is certifying the information from a particular SIR 114a-n. Thus, it is the states or jurisdictions that provide or issue certified documents which, in turn, can be received, stored and transmitted by the record sharing service 120 of vaccination record system 128.

Like everything else, criteria for certification does vary from state to state. The system 100 provides a vault 126 and the ability to transmit immunization records 910 and COVID-19 health status records 2302. The system 100 itself does not provide “certification”-only the States do that. The system 100 merely obtains certified records from the SIRs 114a-n on behalf of our subscribers 602, and store the records 910, 2302 the subscriber's 602 digital vault 126, and then optionally forward the state certified record(s) 910, 2302 to third party requesters 138 on behalf of subscribers 602.

If icon 2010 is selected and other members of the household have been tested, as recommended by message 2006, the screen shot of FIG. 21 is displayed when all members of the household have tested negative, as indicated by message 2102. A status date for the household can be provided as indicated by a message 2106, and an expiration date for the household vaccination status can be provided by a message 2108.

For the “Household Negative Status” of FIG. 21, the tests results are analyzed for the subscriber 602 and profile members 608a-e, and considered cumulatively. The tests for the subscriber 602 and profile members 608a-e should be administered on the same day or within, for example, three days of each other, so that the results are an accurate snapshot of the household at that time.

If each test is followed according to the guidelines and is FDA approved as a reliable test, then the QR code that can be generated by selecting icon 2110 is just a way to provide the test results of all household members (subscriber 602 and profile members 608a-e) to be produced “on demand” for an entire household.

One or more additional instructions or recommendations 2104, 2106, 2108 can also be displayed. Upon receiving the household negative test status for the household, icon 2110 can be selected to generate a new QR code, such as described above with regard to FIG. 9, that includes, for example, the household negative test status indication 2102 and date of the negative test result 2108.

In an embodiment, tests are utilized that are FDA approved or otherwise deemed by the FDA to provide sufficiently accurate test results. The lab that provides the test results, in connection with on-site testing at a lab, can certify the test results or otherwise indicate that the actual result of the test is being transmitted to system 128 for subsequent storage in the vault 126 for the subscriber 602 and profile members 608a-e.

Similarly for in-home tests, the lab that provides the test results, in connection with in-home testing, can certify the test results or otherwise indicate that the actual result of the test is being transmitted to system 128 for subsequent storage in the vault 126 for the subscriber 602 and profile members 608a-e.

Returning again to FIG. 20, if icon 2010 is selected and all members of the household have not been tested, the screen shot of FIG. 22 is displayed. FIG. 22 provides the COVID-19 health status of subscriber 602, as indicated by message 2202 and COVID-10 health status overview 2204 and related information such as a date of expiration 2206.

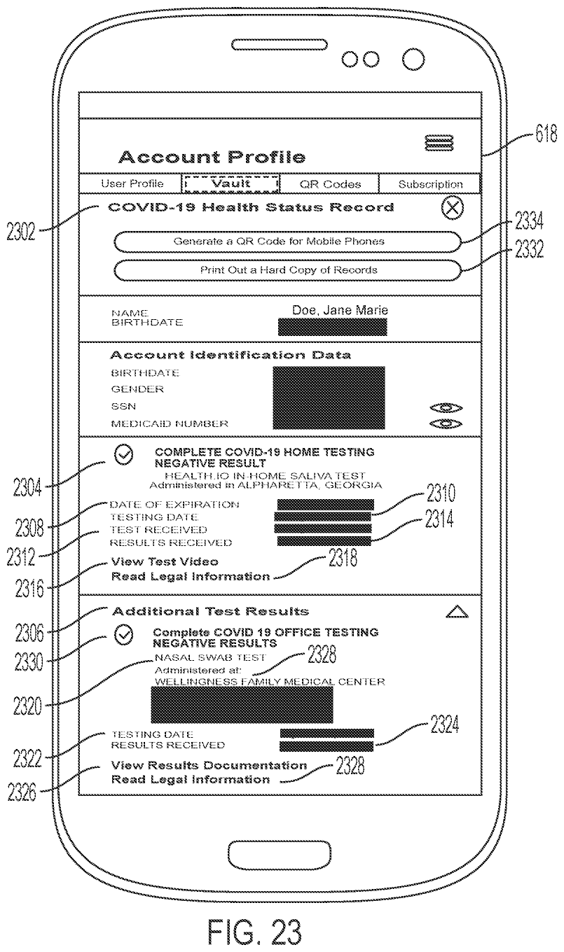

Upon receiving the negative test result for the household, icon 2208 can be selected to view a full certificate of immunization, such as shown in FIG. 10. Icon 2210 can be selected, which allows COVID-10 health status information to be viewed, such as shown in FIG. 23.

FIG. 23 is a screen shot that shows the COVID-19 health status record 2302. The record can include home testing results 2304, and additional test results 2306, such as office testing results 2330. Home testing information can include the data of expiration 2308, the testing date 2310, the date that the test was received 2312, and the date that the test results were received 2314.

The video, as discussed with regard to FIG. 16, can be viewed. Other information such as legal information 2318 can also be provided.

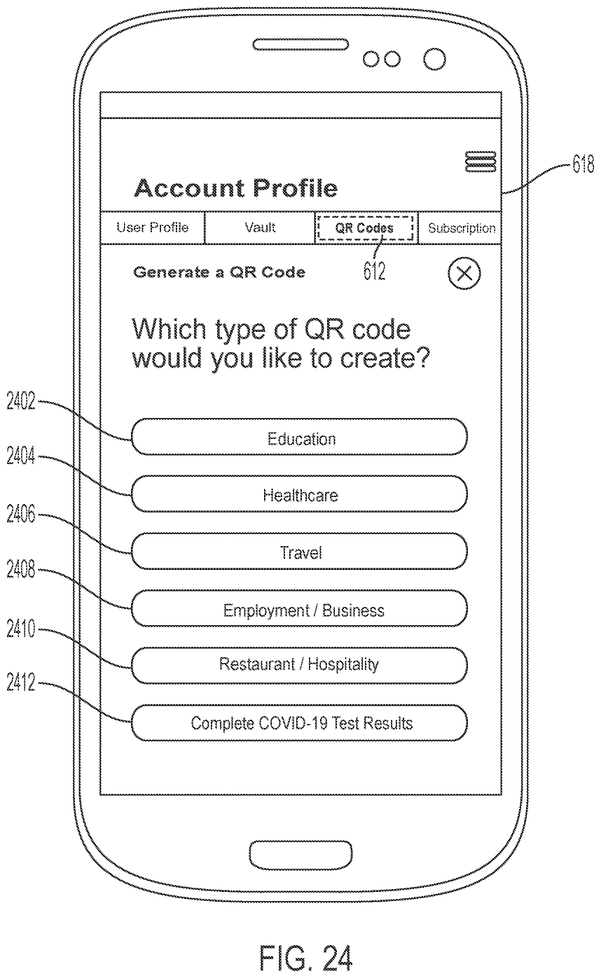

Office testing results 2330 can include the type of test administered 2328, the organization and location that administered the test 2320, the testing date 2322, and the date that the test results were received 2324. Other information such as test results documentation 2326 and legal information 2328 can also be provided. A hard copy of any COVID-19 health status record can be printed by selecting icon 2332. When generate QR code icon 2334 is selected, the screen shot as shown in FIG. 24 appears.

FIG. 24 is a screen shot that allows the subscriber 602 or a profile member 608a-e to select a specific type of QR code. For example, exemplary QR codes are shown for education 2402, healthcare 2404, travel 2406, employment I business 2408, restaurant/ hospitality 2410, and complete COVID-19 test results 2412.

The exemplary QR codes may contain different data depending on the purpose. For example, a restaurant/ hospitality QR code that is generated by selecting icon 2410 may include information such as COVID-19 health status overview 2204 information, such as show in FIG. 22. Alternatively, a healthcare QR code that is generated by selecting icon 2404 may include home testing 2304 information and associated related information 2309-2318, along with additional COVID-19 test information 2306 such as COVID-19 office testing 2330.

FIG. 25 is a screen shot of a healthcare QR code 2502 that generally includes the same information depicted in FIG. 23. That is, in addition to the information included in FIG. 23, FIG. 25 also includes a QR code 2502 associated with the information. In addition, a QR code for use with a mobile phone 106 can be generated in a same or similar manner as described above with regard to FIG. 9. A hard copy of any COVID-19 health status record can be printed by selecting icon 2506.

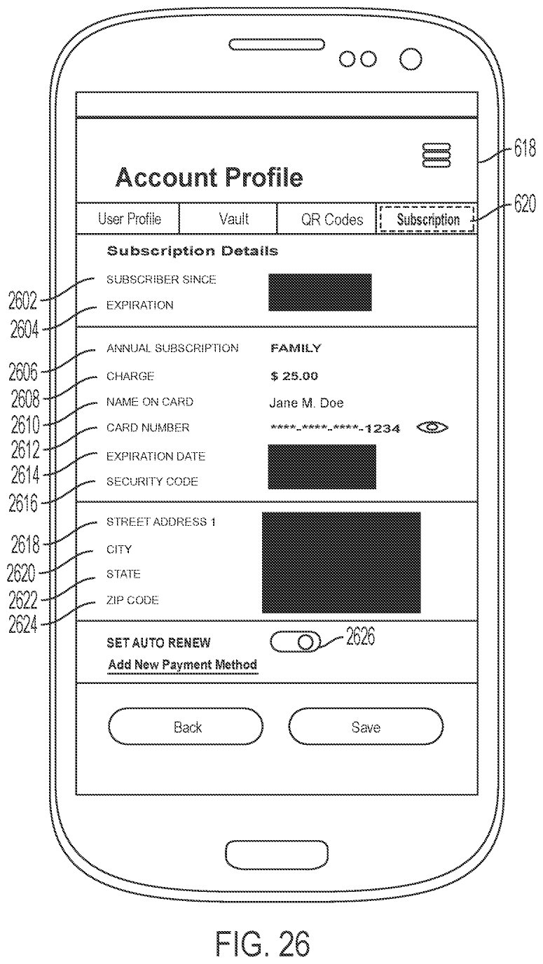

FIG. 26 is a screen shot of the subscription details of a subscriber's 602 subscription details. Subscriber information such as the initial date of becoming a subscriber 2602, the expiration (fee due) date 2604, annual subscription status (individual or family) 2606, subscription charge 2608, name on credit card 2610, card number 2612, card expiration date 2614, card security code 2616, street address 1618, city 2620, state 2622, zip code 2624, and option to automatically renew icon 2626. If the automatically renew icon 2626 is set to “on” status, the expiration (fee due) date 2604 could indicate, for example, “none”, “auto renew”, or display a similar message.

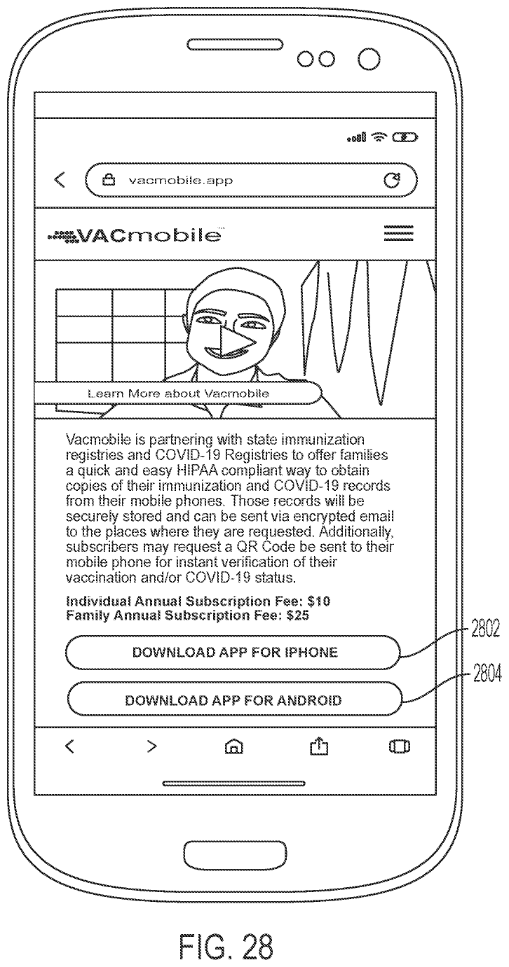

FIG. 28 shows a schematic representation of a user interface that allows an app to be downloaded, by selecting an icon 2802, 2804, for use with the system of FIG. 1. After downloading the app, a user 102 can set up an account. As in initial step in setting up an account, a screen shot such as FIG. 4 will be displayed.

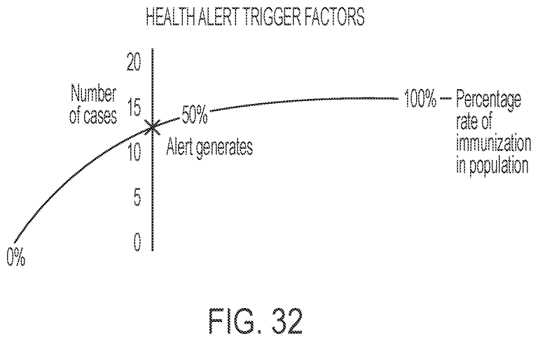

FIG. 32 shows a diagram that illustrates an exemplary and non- limiting manner by which health alert trigger factors can be determined. In the context of predicting a measles outbreak, the system 100 can use AI to identify and analyze subscriber 602 meta data, such as the zip codes where herd immunity (when a large portion of a community (the herd) becomes immune to a disease, making the spread of disease from person to person unlikely) is at risk because the number of vaccinated individual subscribers 602 for measles (or any other disease) has dropped below a predetermined level, because the percentage of subscribers 602 has decreased by a predetermined threshold or amount, and/or because the absolute number of subscribers 602 has decreased by a predetermined threshold or amount over a certain time period (e.g., six months or one year).

Various analytic techniques and/or algorithms can be used to analyze data and deduce possible outcomes that could serve as a first alert for public health officials. For example, FIG. 32 indicates that an alert will be transmitted when the percentage of the population, for one or more zip codes or regions, that is up to date on a measles vaccination is at 45%, and there have been 13 new cases of measles identified within the zip code(s) or region(s) over a certain time period (e.g., six months or one year).

Embodiments of the system 100 also review subscriber 602 records in vault 126, such as shown in FIGS. 7-9, and transmit an alert to the subscriber 602 when a vaccination is due for themselves, a parent, a minor child, or a pet. The system 100 also utilizes and provides an personal assistant capability to assist subscribers 602 with tasks such as scheduling vaccination appointments with their doctors, for themselves, a parent, a minor child or a pet.

The system 100 can also interface with third party systems 138, such as those utilized by insurance companies, to ensure the medical appointments such as annual physical examination appointments and made and completed within the time frame provided for and covered by the subscriber's 602 insurance policy. For example, if the system receives an in indication from a third party 138 that a subscriber's 602 annual benefit for a yearly physical examination expires in July 1, the system can send the subscriber 602 one or more alerts or notifications prior to July 1 that the subscriber 602 should have an annual physical prior to July 1, or the subscriber 602 will lose such benefits for that particular year.

In another embodiment, the system 100 utilizes AI to make various recommendations. For example, if the system 100 determines that a minor child of a subscriber 602 is not due for a vaccination, the system 100 can transmit a message or alert to the subscriber 602 that a tele-health appointment be scheduled rather than an in-person appointment for an annual check-up.

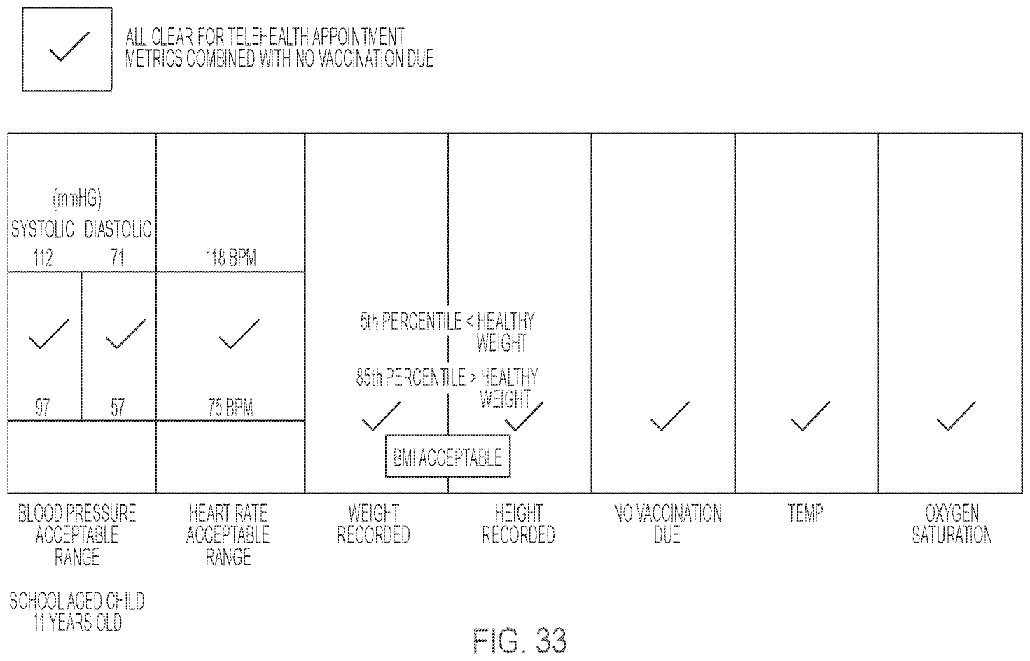

An AI personal assistant can evaluate metrics, as shown in FIG. 33, such as weight, height, blood pressure, oxygen saturation, temperature and heart rate of subscribers 602 and/or their minor children/dependents. Such information can be collected and stored in the vault 126 for the subscriber 602 and/or other profile members 608a-e (dependents). If the subscriber's 602 health metrics fall within a predetermined range, in combination with a “no vaccination needed” immunization status 808, the subscriber's 602 record would trigger an alert or notification for a tele-health appointment in lieu of an in-person appointment.

Guidelines for pediatricians and primary care and family medicine physicians can be obtained from, for example, the CDC's website. According to the CDC website, “growth charts are tools that contribute to forming an overall clinical impression for the child being measured.” System 100 would analyze the subscriber's 602 health information in the vault 126 and compare the information with a range of acceptable data points. If the subscriber's 602 information is within the range, the system 100 will transmit a notification or alert to the subscriber 602 suggesting a tele-health appointment in lieu of an in- person appointment.

Whether the system 100 transmits a notification or alert suggesting a tele-health appointment will generally involve the consideration of a multiplicity of variables and factors, what can vary from region to region, and over time. For example, tele-health appointments for annual physicals may be preferred if it limits subscriber's 602 exposure to infectious diseases. Reducing in-office burdens on medical personnel, thereby allowing them to serve a greater number of patients, can also be a consideration.

Additionally, acceptance of tele-health annual physicals as a best practice this could maintain standards and advantageously improve health outcomes. Subscribers 602 may be less inclined to postpone or forego medical appointments for themselves or their children, for example, to reduce the chances of catching an infectious disease.

The current COVID-19 crisis has shown that deaths have occurred as a result of postponing or foregoing medical care or appointments that could have addressed symptoms before the condition worsened. An example of a technology assisted innovation that was unheard of when it was introduced but has now become a worldwide best practice is Project ECHO (Extension for Community Healthcare Outcomes) is a collaborative model of medical education and care management that empowers clinicians everywhere to provide better care to more people, right where they live.

The ECHO model does not actually “provide” care to patients. Instead, it increases access to specialty treatment in rural and underserved areas by providing front-line clinicians with the knowledge and support they need to manage patients with complex conditions such as: hepatitis C, HIV, tuberculosis, chronic pain, endocrinology, behavioral health disorders, and many others. It does this by engaging clinicians in a continuous learning system and partnering them with specialist mentors at an academic medical center or hub.

As the ECHO model expands, it is helping to address some of the healthcare system's most intractable problems, including inadequate or disparities in access to care, rising costs, systemic inefficiencies, and unequal or slow diffusion of best practices. Across the United States and globally, policymakers are recognizing the potential of ECHO to exponentially expand workforce capacity to treat more patients sooner, using existing resources. At a time when the health care system is under mounting pressure to do more without spending more, this is critical.

The ability to connect highly specialized professionals directly via telehealth (the distribution of health-related services and information via electronic information and telecommunication technologies, which allows long-distance patient and clinician contact, care, advice, reminders, education, intervention, monitoring, and remote admissions) with local frontline healthcare workers, doctors and nurses for the treatment of complex conditions for their patients has greatly improved health outcomes and become a worldwide best practice.

Likewise the ability for a patient to know if a shot is needed made possible by the data stored in the vault 126. Access to this data in the vault 126 will decrease the burden now placed on frontline primary care doctors to physically see patients and children in their offices when a tele-health visit is preferable.

In another embodiment, the system 100 determines if a subscriber's 602 physical address has changed by periodically generating an address update check, such an annually. Subscribers 602 can respond, and indicate whether the subscriber's 602 physical address has changed, or remains unchanged.

If the subscriber's 602 address has changed, as verified by the subscriber 602, the system 100 would begin searching the SIR 114a-n that corresponds the updated address of the subscriber 602. The system 100 would then check the vaccination requirements for the subscribers 602 new state in which the subscriber 602 resides, to ensure the subscriber's 602 vault 126 is up-to-date. That is, the system 100 will provide the subscribers 602 vault 126 records to the SIR 114a-n of the new state in which the subscriber 602 resides. The system 100 can query subscribers 602 periodically with regard to the current address, in order to perform the functions described above.

In another embodiment, the system 100 utilizes GPS technology to track when a subscriber travels to another state (FIG. 8, 806). For example, suppose a subscriber 602 resides in Georgia, travels to Florida, visits a pharmacy in Florida, and receives a flu shot at the pharmacy. The system 100 will then initiate a search of the visited state's (e.g., Florida) SIR 114a-n and update the Florida SIR 114a-n with vaccination records from other states in the subscriber's 602 vault 126 record. The system 100 will also update the SIRs 114a-n of which the subscriber 602 currently resides (e.g., Georgia), indicating that the subscriber 602 has received a flu shot in Florida. Thus, the SIRs 114a-n of all relevant subscriber 602 states will contain the same vaccination record information for the subscriber 602.

The system 100 may also use AI to predict that subscribers 602 may be planning for foreign travel when obtaining certain vaccinations that are required for travel to foreign countries and may automatically offer subscribers assistance with additional tasks such as auto-filling paperwork required by an embassy or consulate and scheduling virtual appointments with consulates or embassies for the preparation of travel visas. By reading and analyzing subscriber 602 information in the vault 126, it can be inferred that the home address of the subscriber 602 may have recently changed, which thereby triggers the system is transmit questions regarding possibly travel, or to confirm the subscriber's newly entered address.

In addition, embodiments of the system 100 may also assist subscribers 602 with tasks such as sending follow up emails with human resources departments when working with employers. A virtual assistant can be used to anticipate the reason for a subscriber's 602 vaccination record request based on the location the vaccination is being sent to (see FIG. 29) and provide recommendations and/or assistance in connection with potential activity that might follow. For example if one child's vaccination records stored in the vault 126 are being transmitted to a school, the system can ask the subscriber 602 if they would like other vaccination records stored in the vault 126 for children to be transmitted to the same school or a different school. The organization (e.g., school or other organization) may generate a list (or other data format) of all individuals attending an event (e.g., a concert, a school day, a school event, etc.). The list may include information regarding each individual, such as, for example, a role of the individual, a quarantine status of each individual, a start/end date of each quarantined individual, a vaccination status of each individual, whether each user has reported an encounter or contact with an infect individual, timing information such as when a vaccine was received. The list may include information as to whether the individual is presently known to be at the organization. As an example, the system 100 may receive a notification when a user arrives at an event or location of an organization and a notification when a user leaves the event or location (e.g., a check-in or check-out event when a person comes or goes).

In some embodiments, the notification can be generated automatically based on a geolocation of each individual entering or leaving a geofence around the event or location. The system 100 can render information from the list on a display for review by the organization. The system 100 can aggregate data from the list of individuals and provide a dashboard with various details, such as overall vaccination rate of individuals at an event or location, exposure history or status of individuals at the event or location, quarantined ratio of individuals, demographic information, etc.) In some embodiments, the system 100 can receive configuration of parameters regarding the aggregated data for an event or location and enforce those configured parameters. The enforcement can occur during a check-in and check-out process at the event or location. As an example, the system 100 can receive a check-in request for each user (e.g., by scanning a QR code 902 of the user, by having the organization indicate the user is checking in, by having the user enter a geofence, by scanning an RFID tag associated with the user at a check-in location, by receiving an indication that a ticket assigned to the user has been scanned or redeemed, etc.) and determine whether to admit the user based on aggregated user data for the event.

In one example, the organization may configure the parameter to require 90% of users in attendance be vaccinated. In this example, the system 100 can determine 100 people are currently attending the event and 95 are vaccinated, and deny access to a new person at check-in based on the individual being unvaccinated (or the status of the individual's vaccination is uncertain or unknown). Further to this example, if an unvaccinated per left the event or nineteen vaccinated individuals checked in (e.g. the ratio would still be within the threshold if the unvaccinated person was admitted), the system 100 can admit the unvaccinated person to the event. In some embodiments, the system 100 can generate a queue of individuals waiting for admission where admission would cause the individuals to cause a value of the parameter to exceed (or recede) past a configured parameter threshold (referred to herein as fail to meet the parameter threshold for simplicity). Once attendance at the event or location changes where admittance would not cause the attendance to fail to meet the parameter threshold, the next individual on the queue can be admitted. In some embodiments, multiple queues can be generated for different parameter thresholds.

In some embodiments, the system 100 can control registration for an event based on parameter thresholds. As an example, the system 100 can limit purchasing of a ticket to an event or registration for a class at a school if doing so would cause failure to meet a parameter threshold. The system 100 can similarly maintain a queue of purchasers of tickets or registrants for a class (or other event) for when the person would not cause the failure. As an example, the system 100 can be configured to require 80% of the class being vaccinated and the system 100 can grant or deny registration to the class based on a vaccination status of each individual user that registers data for currently registered users.

Embodiments of the system 100 may also utilize or interface with contact tracing software to ensure that survivors of COVID-19 are made aware that they can keep their COVID-19 health status records in the vault 126. If a subscriber 602 has been notified of exposure to COVID-19 , the system can send a message to the subscriber 602 indicating that the system 100 can obtain, store, and transmit the subscriber's vaccination records and/or COVID-19 test results to third parties 138 at their request. The vaccination record system 128 can receive an indication from a particular user 102 that the particular user 102 has been in contact with someone that has a particular disease or that the user 102 has contacted the particular disease. The vaccination record system 128 can determine a list of individuals to contact to inform the individuals that they may have been in contact with someone associated with the particular disease. As an example, the vaccination record system 128 can determine a list of users 102 that have been proximate to the particular user 102 based on GPS data from the a user device (e.g., a mobile phone 108, a tablet 110, etc.). The particular user 102 may select one or more contacts from their contact list or enter a list of individuals to contact regarding the potential exposure.

Once a list has been determined, the vaccination record system 128 can generate an alert to the individuals in the list to notify the individuals that they may have been exposes. The notification can be sent via an email, text, phone call, mail, or through some other means. In some embodiments, the vaccination record system 128 can determine that a subset of the individuals are users in the user registry 124. When an individual is in the user registry 124, the vaccination record system 128 can initiate another tracking of the individual. In one embodiment, the vaccination record system 128 can determine which of the users in the user registry 124 have been vaccinated against the particular disease. When vaccinated, the vaccination record system 128 can remove the users from being notified or notify the users with a different message (e.g., a message that confirms the user is vaccinated already).

If the user gives permission, the vaccination record system 128 can initiate compiling a second list of individuals that have contacted the user. In some embodiments, the second list of individuals can be generated immediately, while in other embodiments, the vaccination record system 128 can await for the results of a test for the particular disease to generate the list only if the user tests positive for the particular disease. In other embodiments, the vaccination record system 128 can request confirmation that the individual has one or more symptoms associated with the particular disease before initiating the notification of contacted individuals. When an individual is not in the user registry 124, the vaccination record system 128 can send a request to the individual to enroll in the vaccination record system 128 as a user in the user registry 124. In some embodiments, the vaccination record system 128 can mark one or more users in the user registry 124 as having been in contact with someone having the particular disease and initiate a quarantine window. During the quarantine window, the vaccination record system 128 can limit or alter a certificate of immunization or health status for a particular disease to indicate the potential exposure. In some embodiments, the vaccination record system 128 can revoke a certificate of immunization or change a health status in response to the exposure. In some embodiments, an organization can limit admission (while others may not) based on the potential exposure such scanning of a QR code for a fully vaccinated person may result in the person being denied admission by the system 100 during the quarantine window.

Embodiments of the system 100 may also assist government bodies or agencies to use geofencing (the practice of using global positioning (GPS) or radio frequency identification (RFID) to define a geographic boundary) capabilities to transmit municipality specific alerts (such as from a Mayor or Governor) that include, for example, information about COVID-19 testing sites, or guidelines from the CDC or their local Government. This alert can be considered to be similar to the use of standard “amber alerts” (an emergency response system that disseminates information about a missing person (usually a child), by media broadcasting or electronic roadway signs).

Embodiments of the system 100 may also be used for mobile medical alert capabilities for First Responders and emergency room (ER) personnel to access a subscriber's 602 vault 126 to review their vaccination records with prior member permission for emergency situations. The subscriber 602 signs an agreement that authorizes a First Responder or an Emergency Medical Doctor or Nurse to access their vault 126 in an emergency situation. An Emergency Code would be entered in the subscriber's 602 mobile phone 108, for example, to open the subscriber's 602 vault 126. The First Responder or ER licensed physician or nurse would then input information such as their name, their employer, the reason(s) for accessing the subscriber's 602 vault 126, and date and time of review of the records. For example, a First Responder could arrive at a subscriber's 602 home and find a subscriber 602, such an elderly person, in severe respiratory distress. The first responder could immediately review the subscriber's 602 immunizations records 910 and COVID-19 health status records stored in vault 126.

Embodiments of the system 100 allow subscribers 602 to store their genetic code sequencing stored in their vault 126 and have the capability of sending it or producing it on-demand in a medical appointment to assist a medical professional when they are identifying the best treatment of a particular disease. It has been well documented that scientists have theorized that the genetic sequencing of an individual is related to the high degree of variability in the symptoms that individuals show when they contract COVID-19 . A subscriber's 602 genetic code may be utilized to determine which type of vaccine would be the most effective for that subscriber 602.

The system 100 can also provide subscribers 602 with the ability to have their emergency medical records (EMRs) stored in their vault 126, and not just immunization records 910. The system 100 thus provides portability of EMRs and interoperability with third party systems to allow different systems to interface with each other.

For example, if a medical professional has a patient in a hospital that uses Cerner as an EMR platform, that the medical professional is not generally able to access information from a hospital that uses Epic as an EMR platform. The system 100 facilitates communication between disparate systems that previously were not able to exchange information with each other.

For example, the system 100 can use Java, which is known computer software and specifications that provide a system for developing application software and deploying it in a cross-platform computing environment.

The system 100 may also provide customized QR codes. For example, a QR code can pertain, for example, vaccination status and information that pertain to a particular country (e.g., India), a sporting event, other events open to the general public, and/or private establishments such as a restaurant. For purposes of travel, QR codes can be tailored to demonstrate that an subscriber 602 has met or is in compliance with the required vaccinations and or COVID-19 health status (or other communicable disease health status) requirements of a particular country or State they are traveling to.

Currently, in the U.S., certain states are requiring incoming visitors to quarantine for 14 days upon arrival. A customized QR code for such incoming visitors could indicate that the visitor has, in fact, met such requirement(s) in order, for example, to be able to enter spaces such as grocery stores, restaurants, apartment complexes etc. Customized QR codes can also be used, for example, by employees of the U.S. State Department, airline carriers, and airports.

The system 100 may also use custom QR code scanners or biometric scanners for large-scale congregate events, restaurants, schools, employers there may conclude that the current survey questionnaire method of screening entrants to various life situations including returning to work, school or extra-curricular activities is not the most effective method of screening.

There may be a desire to more scientifically screen who can and cannot enter the business or establishment. Some places may choose to have open source QR code reader apps on an entry guard's cell phone that will read the customized QR code. However, some businesses may prefer an actual large piece of hardware such as a scanner that individuals interact with when entering an establishment such as a stadium or an airport.

The system 100 can also be used in situations where subscribers 602 need to show that their entire household is up to date on required immunizations, as well as any additional health status tests. For example, a subscriber may have a QR code for their child or other minor stored on their mobile phone 108, so that the QR code can be scanned by a bus driver before the child enters the school bus or before the child exits the car in the carpool line at pick-up and drop off locations at public schools and secondary educational institutions.

Similarly, the system 100 can interface with hospitals and healthcare providers to have the up-to-date immunization record and clean bill of health household QR code scanned as the subscriber 602 enters a hospital. Similarly, a hotel or apartment complex may require their guests or tenants (subscribers 602) to have QR codes on their mobile phones 108 scanned at the entrance to the complex to ensure that the subscriber 602 is up to date on their immunizations and/or COVID-19 health status required tests.

Private employers may pay for their employees (subscribers 602) to have access to testing that is more comprehensive than what a municipality requires. The system 100 can interface with private entities to ensure all types of testing results can be captured in the subscriber's 602 vault 126, and optionally transmitted to or otherwise shared with third parties 138 or produced on demand via a QR code.

In various embodiments, exemplary functionality of a vaccination record verification system and method is shown herein for storing, transmitting, and authenticating vaccination records. The vaccination record verification system may utilized by an organization to validate the vaccinations and/or vaccination record of a registered user.

The vaccination record verification system may include a software application, cloud based storage, network communications, APIs, and other systems applications as described herein. Additionally, the system may include geo-location functionality or communications with a third-party to provide geo-location information for computing devices, such as a mobile phone 108, desktop or laptop computer 106, and/or tablet computer 110 (FIG. 1). Also, the system may read, communicate, or interface with a third-party's website, or scrape vaccination data from a third-party's website, as described below with regard to verifying a user's vaccination record. Certain functionality may be discussed with respect to the application as executed on a computing device; however, it is understood that this functionality may be performed wholly or in part in other computing devices, such as, for example, in or in coordination with a record retrieval service, a record sharing service, an enrollment service, or other services provided by the system.

A user may download the application onto a computing device, such as a mobile phone, tablet, personal computer, etc. In one embodiment, an organization may provide the user with an organizational code. The organization may include a travel or hospitality service provider. The user may be a user of the organization's services or an employee of the organization. The organization may provide the organizational code to the user via email, loyalty website registration, convention or event registration, point of sale systems, social media, and other similar methods. The organizational code may be utilized by the user to facilitate downloading the application and allowing the organization to access the user's vaccination records at an appropriate time.

The user may create an account and the application may prompt the user to verify or authenticate the user's identification. The user may input account information into the application as described elsewhere in the present disclosure. The application may include one or more methods of authenticating or verifying the user's identification, including uploading an image of the user's government issued identification card via a camera function on the computing device, initiating a video chat with a customer representative, verifying details about a user from an information data base, providing questions to the user to verify user information, calling or texting a mobile number known to be associated with the user, or other verification steps. The application may also verify the user's identification by comparing the account information input by comparing received vaccination records to the user's account information (e.g., compare the name on the vaccination record to the name of the user of the account). The user's identification and account information may be stored in the HIPAA-compliant cloud storage. The user authentication process may also include two-factor authentication as described elsewhere in the present disclosure.

The user may make an appointment to receive a vaccination or a first dose of a multi-dose vaccination with a vaccination provider. The user may travel to the vaccination provider location to receive the vaccination or first does of a multi-dose vaccination. The application may utilize geo-location functionality to determine and record/store the location of the computing device (e.g., the user's location) and the date and time the device was at the location. The application may utilize geo-location functionality to determine that a user was at a certain address or at certain coordinates at a certain date and time, and may further determine location information based on the certain address or certain coordinates. The system can compare the location of the user to a location of a doctor registered as providing the vaccine to verify the user received the vaccine.

Vaccination doses may include identification numbers on vials and/or syringes, such as, but not limited to, a manufacturer number and a lot number. The manufacturer number may refer to the company that produced the vaccination dose. The lot number may refer to a specific batch of vaccination dose produced by the manufacturer. The manufacturer and lot numbers may include a scannable bar code, QR code, or other similar type of code that provides the manufacturer and lot numbers to a computing device once the computing device scans the code. The application may receive the manufacturer and lot number as proof of vaccination from the user. The application may receive or capture a digital image (via image-capture functionality of the computing device) of the manufacturer and lot numbers of the vaccination, receive a scan of the manufacturer and lot numbers of the vaccination, and/or receive a manual input from the user of the manufacturer and lot numbers of the vaccination, pull the manufacturer and lot numbers from a database based on a code obtained from the digital image of the vaccine, or other similar input methods so that the application receives the manufacturer and lot numbers of the vaccination dose that the user received. The application may receive the proof of vaccination while the user is at the vaccination provider location, or may receive the proof at some other location if the user manually inputs the manufacturer and lot numbers of the vaccination dose at a later time. As an example, the application may receive proof by capturing an image of a vaccination record or verifying a geo-location of the computing device is within a geofence proximate a medical facility giving the vaccine. In one embodiment, the application may receive a request to verify the vaccine, transmit the request to the system, and the system may transmit a request for confirmation from a user at the doctor's office. The application on either the user's or the doctor's office user's computing device may scan a code on the other user's computing device to verify the vaccine was given to that patient. The application may also receive a digital image of the vaccination record provided by the vaccination provider (e.g., CDC White Card) as proof that the user received the vaccination dose. The application may utilize handwriting recognition functionality or image/word recognition functionality to read and record the vaccination information, manufacturer, and lot number. The application may assign a date and time that an image was captured as the date and time the user received the vaccination dose. The application may receive the date and time of the vaccination dose via manual input from the user. The user may repeat the above process if the vaccination requires multiple doses or if multiple vaccinations are given.

For example, a user may go to vaccination provider, such as a pharmacy, doctor's office, hospital, etc., to receive the first COVID19 vaccination dose. At the vaccination provider, the user may scan a QR code on the vaccination vial to capture the vaccination dose manufacturer and lot number. In some embodiments, the vaccination vial is scanned by a nurse or doctor, such as, for example, for compliance reasons. The application may receive the QR code scan and capture/receive the manufacturer and lot numbers of the vaccination dose, and store the information in the HIPAA-compliant cloud, application (e.g., on the device storage), or in other similar secure storage. The data may be stored using encryption-at-rest technology to prevent theft of the data. The application may record the location of the device (e.g., the user's location) at the time the application received the QR code. The application may link the manufacturer and lot numbers of the vaccination dose with the date and time the user received the vaccination and the location of the vaccination provider where the user received the vaccination. Since some vaccinations (e.g., COVID-19 vaccines) are multi-dose vaccinations, the user and application may repeat the above steps if the user receives a second or subsequent vaccination dose.

The application may verify the user's vaccination by verifying that the vaccination dose the user received was physically at the vaccination provider location at the time the user received the vaccination dose. The application can access a manufacturer's vaccination information, from a third-party's website or service, such as a manufacturer's website, the Center of Diseases Control's (CDC) website, or other similar website that has the manufacturer vaccination information. The application may also have access to information received from the manufacturer or CDC (e.g., the manufacturer may provide confidential vaccination lists to the application), or may create internal lists with the manufacturer vaccination information by querying a web or network service, or scraping the manufacturer's website, the Center of Diseases Control's (CDC) website, or other similar website for the manufacturer vaccination information data and compiling tables for the data. The application may query an API provided by the manufacturer to determine The application may scrape the manufacturer's website for The manufacturer's vaccination information may include a list of all manufactured vaccination doses, the manufacturer number and the lot number for each of the doses, a date of manufacture and a shipment date, and the shipment location for each vaccination dose. The shipment location may be the vaccination provider location, and the shipment date may be the date the vaccination dose was shipped to the shipment location and/or the date the vaccination dose was delivered to the shipment location. The application may have functionality that searches the manufacture's vaccination information for the manufacturer and lot numbers of the vaccination dose received by the user in the manufacturer's vaccination information. The application may then receive the shipment location and the shipment date for the vaccination dose matching the manufacturer and lot numbers from the manufacturer's vaccination information.

The application may compare the shipping date to the date the user received the vaccination dose. If the shipment date and the date the user received the vaccination are within an acceptable range, the application verifies that the user received the vaccination dose. The acceptable range may be the time between vaccination dose production and expiration date of the vaccination dose, and may be set by a third party, such as the CDC or other regulators, or the manufacturer. The acceptable range may be a specific amount of time, such as, but not limited to, four weeks, ninety days, etc. The application may identify one or more discrepancies in the vaccines and request a remedial action be performed or deny the vaccination dose being assigned to the user. As an example, application may determine that the shipment date is subsequent to the vaccination dose date and perform a remedial action, such as, for example, flag the vaccine information for additional review or deny the vaccine being assigned to the user account.

The application may then compare the shipment location for the vaccination dose having the same manufacturer and lot numbers to the recorded geolocation of the user at the date and time the user received the vaccination dose. If the recorded geolocation of the user at the date and time the user received the vaccination dose is the vaccination provider location, the application verifies that the vaccination dose was at the vaccination provider the user went to receive the vaccination. The application may determine that the geolocation does not match the shipment location and perform a remedial action.

In another embodiment, the shipment location may be a government agency responsible for distributing the vaccination doses to various vaccination providers, such as a state, city, or county, federal, or other similar agency. In this embodiment, the application may access the distribution list of the government agency so that the application may compare the manufacturer's vaccination information, the government agency distribution list, and the recorded vaccination provider location. The application may compare the government agency shipment location to the recorded vaccination provider location for verifying the user's vaccination dose. For example, the shipment location to city agency from the manufacturer may be Atlanta, Ga., and the recorded vaccination provider location that the user received the vaccination dose at may be in Atlanta, Ga., and the application may verify the user's vaccination because the shipment location and the vaccination provider location included the same address (or addresses in close proximity), the same city (or cities in close proximity), or the same state.

In certain embodiments, multiple manufacturers may provide vaccinations against the same disease. In this embodiment, the application may compare the manufacturer number from the user's vaccination dose against the manufacturer number of each of the manufacturers to verify the correct vaccination dose manufacturer. The application may then proceed with the verification process as described above.

Once the application has verified the user's vaccination dose(s), the application updates the user's account to indicate that the user has received the vaccination. The application may send vaccination information to the SIR so that the SIR may be updated. The application may send the updated vaccination information to vaccination registries. The application may create a vaccination registry that includes the users of the application and the users' vaccination record information.

The application may also generate a user interface that provides information that one or more vaccines have been received, which can be referred to as a health pass. The health pass may include a scannable identifier, such as a QR code, on the user interface of the user's computing device, immunization/vaccination records, and user identification information. An computing device of an organization or individual may scan the scannable identifier on the health pass to determine if the user has had certain vaccinations. Upon scanning of the scannable identifier, the application may cause a new screen to form on the user interface that indicates whether the user has had the certain vaccinations. The organization may then allow the user to utilize the organization's services or not, depending on if the user had had the certain vaccinations. The application may analyze a vaccine history of the user before generating the user interface. The application may determine whether a vaccine is past due according to a current date and update the user interface. As an example, for a user that received a tetanus vaccine on Apr. 1, 2010 with a second dose due in ten years, the application may render a user interface indicating that the user is current on the vaccination for tetanus on Mar. 30, 2020 but that the user is not current on the vaccination for tetanus on Apr. 2, 2020.

As an example, a gym, grocery store, library, school, government building, or other facilities or events may require a valid health pass be scanned prior to allowing entry into the facility. Other examples of facilities and events include spas, concerts, flights and travel, cruises, restaurants, clubs, parties, reunions, sporting events, conferences, vacations, trade shows, and other meetings, facilities, and events. For example, an organization may not allow anyone to access their services that have not had the COVID19 vaccination. If a user tries to access the organization's services, the organization may scan the scannable identifier on the health pass on the user's application, which may cause a new screen to appear that indicates that the user has had the COVID19 vaccination. The organization may then allow the user to access the organization's services. Some other specific examples of organizations or facilities that may scan a user's health pass include, but are not limited to, airports/airlines, entertainment venues, hotels, conventions, cruise ships, etc. In some embodiments, the facility or event may include a scanning device at an entrance for a user to scan the health pass. For example, a user may walk into a subway station and scan the health pass at a scanner which validates the health pass and unlocks an entrance for the user if validated, such as a turnstile. The application may also alter one of more settings of the computing device while presenting the health pass and reverse the setting changes once the health pass is no longer shown. As an example, the application may increase a brightness of the display, enable a do not disturb mode, prevent popups on a display, lock an orientation of the display, or perform other changes when the health pass is being displayed. The changes may facilitate the health pass being scanned by a scanning device.

In another embodiment, the organization may have exclusive access for users that verify, via the scannable health pass process, that the user has received the required vaccinations (“verified vaccinated users”). The exclusive access may be for access to an entire facility or to certain areas of a facilities. For example, a cruise ship or a hotel may have designated vaccination-safe floors, restaurants, and/or bars for the verified vaccinated users, an entertainment venue may have exclusive access to designated areas for verified vaccinated users, and/or a convention center may grant exclusive access to certain meeting rooms to verified vaccinated users. Restaurants may have separate seating for vaccinated individuals from non-vaccinated individuals or individuals with unknown/unverifyable vaccination histories. Some facilities may require masks be worn or other personal protective equipment (PPE) be worn when vaccination histories ad unknown/unverifyable or when an individual is unvaccinated, whereas individuals displaying a valid health pass may be allowed without PPE. In some embodiments, once an individual's health pass has been verified, the facilities may distribute indication devices, such as an armband, a sticker, or some other indication device, to indicate that the individual is properly vaccinated.

Organizations may also utilize the application with an organizational account. The system may provide a user associated with an organizational account to user information and vaccination information for users associated with the organization. For example, the system may provide access to the user and vaccination information for the company's employees. The system may provide a school with access to user and vaccination information for staff and students. The user information may be the user's name, home address, age, race, sex, role in organization, and other similar user information. The vaccination information for the users associated with the organization may be dates and outcomes (e.g., positive or negative) of tests for a certain diseases (e.g., dates and outcomes of COVID19 tests), dates of vaccinations for certain diseases, next scheduled date for a user to receive a test for a certain disease, reported exposure information, quarantine type, quarantine start and end dates for users that have tested positive for a certain disease, and other similar vaccination information. The application may render an organizational dashboard that provides a summary of vaccination information for the organization and the users associated with the organization. Types of information on the organizational dashboard include, but are not limited to, current number of users quarantined, number of users associated with the organization (e.g., employees), number of vaccinated users associated with the organization, number of users currently positive for a specific disease (e.g., COVID19), data tables displaying vaccination information for individuals associated with the organization, graphs displaying vaccination information for users, and other similar data. The data tables may be filterable. The rendered dashboard may include various charts and graphs to illustrate the data.

In some embodiments, the system may collect additional data on users. As an example, the application may receive an input from the user indicating that the user has contacted COVID-19. The system may store data, which may be de-identified, indicating that the user contacted COVID-19 along with the vaccination status of the user. As an example, the system can store the data using a record identifier that does not include any personal information. The system can aggregate records of de-identified data to track health information across multiple individuals or a population of individuals. As an example, the system may track effectiveness rates of vaccines using the data, infection rates in a population of users, likelihood of symptoms and other factors. In one embodiment, the system can receive feedback from various users as to symptoms of receiving a vaccine. The system can utilize de-identified data from a populace of users to provide data to users considering whether to get a vaccination. When a vaccination includes multiple stages, the data may track a current stage in the process of vaccination of the user when an infection occurred. As an example, the system may track that an individual contacted COVID-19 twelve days after receiving a first vaccination of a two-vaccination dose but before receiving the second vaccination. The system may track statistical data including a number of users for an organization (e.g., employees, students, users, etc.), a number of vaccinated users at the organization, a percentage of vaccinated users, a percentage of disease positive users, a percentage of disease negative users, a number of users that have antibodies to a particular disease, such as COVID-19 , and other data. The system may render one or more user interfaces that displays the statistical data, such as a dashboard, for an administrative user or other individual. The dashboard may include details of all users currently at, that have accessed a facility of event, that are registered for, or associated with a particular facility or event. The details may include whether a user is in quarantine, a type of quarantine, when the quarantine started, a role of the user, a most recent test date, a most recent test result, a history of text results, a most recent antibody test, an antibody status, an antibody expiration date, and other information. The dashboard may include a demographic and location insights along with other analytics.

In some embodiments, an organization may create one or more rules or policies for the system to enforce. For example, the organization may generate a rule in the system of specific vaccines that individuals must have before being able to enter a facility. The organization may generate a rule to specify customized quarantine timelines, and the system may enforce the rule to determine whether a user may enter a facility or event by analyzing data when health passes are scanned. As one example, a rule may specify that a specific room or area is restricted to vaccinated users during a particular time window. If a non-vaccinated user scans a health pass outside of the particular time window, the user may be omitted but the user may be denied entry during the particular time window.

In one embodiment, the system may enforce the rules within a facility even after users have been admitted into the facility. As an example, the system may apply a policy to determine that a non-vaccinated user may enter the facility but may not access a particular area. The application may identify that the non-vaccinated user has entered a geofence associated with the particular area and the system may perform one or more remedial action. The remedial action may include generating an auditory, visual, or other alert, sending a communication. A user account associated with the organization may receive a notification that the non-vaccinated user has entered an unauthorized region so that security can remove the user. The system may identify other users within the geofence and generate a warning to those other users that the non-vaccinated user has entered the area.

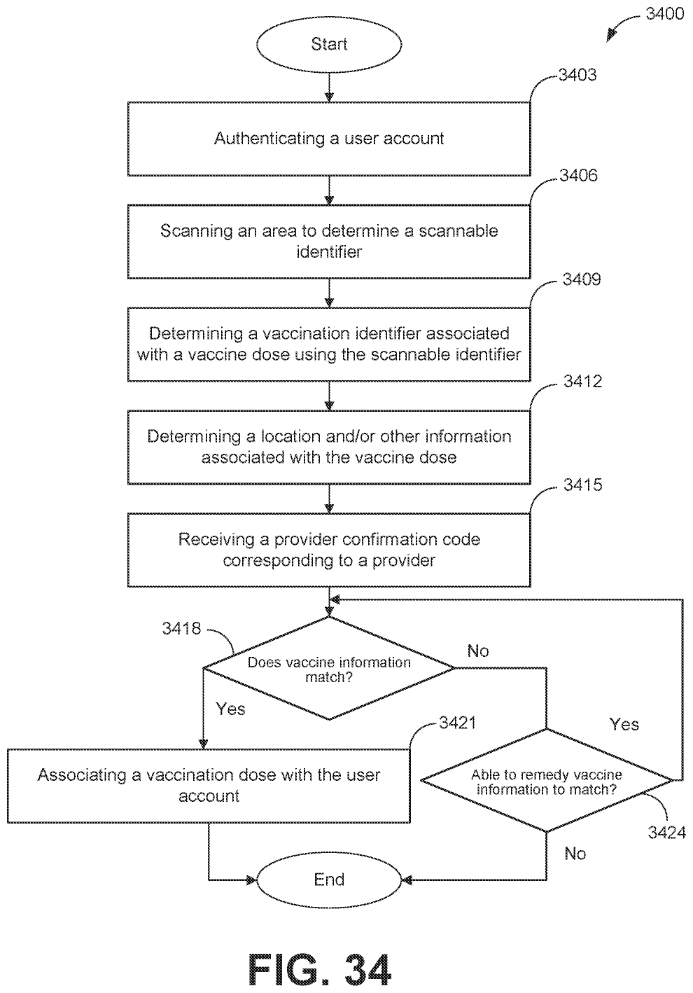

Turning to FIG. 34, shown is a flowchart of a process 3400 according to various embodiments of the present disclosure. In some embodiments, process 3400 can be performed by one or more computing devices such as, for example, a mobile phone 108, desktop or laptop computer 106, tablet computer 110, a vaccination record system 128, a cloud 134, or another system described herein.

At box 3403, the process 3400 can include authenticating a user account. For example, vaccination record system 128 can authenticate a user account against a user registry 124. As an example, the vaccination record system 128 can receive an authentication credential, such as a username and password, a secured token, or other credentials and validate the credential against a user record in the user registry 124. The vaccination record system 128 can receive biometric data and authenticate the biometric data. In some embodiments, the vaccination record system 128 can perform multi-factor authentication. As an example, the vaccination record system 128 may authenticate a credential of the user and transmit a text message (or email, provide via a phone call, generate in an app, etc.) with a random string to a phone number associated with the user account. The vaccination record system 128 can authorize the user to access the account if the random string is entered by the user. The vaccination record system 128 can validate one or more factors of credentials before allowing access.

At box 3406, the process 3400 can include scanning an area to determine a scannable identifier. For example, process 3406 may be depicted by the embodiment in FIG. 15, where the mobile phone 108 (FIG. 1) is used to scan an image to determine the scannable identifier on the vaccine dose. A camera of a mobile phone 108can capture the scannable identifier, for example, from a dose of the vaccine or a certificate or paper. The user may entering the scannable identifier's identification code manually into the mobile phone 108 via a keyboard or voice command. The mobile phone 108 can receive the identifier wireless via communication with a remote device, such as, for example, using near field communication (NFC), passive radio frequency identification (RFID), active RFID, Ultra Wide-Band (UWB) real-time location system (RTLS), WiFi RTLS, infrared RTLS, and Bluetooth. The mobile phone 108 may include a reader device or transceiver capable of querying for and/or reading data via wireless communication. The mobile phone 108 can scan an area including a vile of the vaccination dose. The vile may include any vessel that contains the vaccination dose planned to be used for the user 102. This may include, but is not limited to, a vile, a prepackaged syringe, and a large airtight jar. In some embodiments, the scannable identifier can correspond to a vessel containing multiple doses of the vaccine.

At box 3409, the process 3400 can include determining a vaccination identifier associated with a vaccine dose using the scannable identifier. Vaccination identifier may include, but is not limited to, a batch number and a lot number of the corresponding vaccine dose or set of vaccine doses in a container.

At box 3412, the process 3400 can include determining the location and/or other information associated with the vaccine dose based off of the scannable identifier. The vaccination record system 128 may request information regarding the vaccination does from a third party (e.g., the CDC, a vaccine manufacturer, a distributor, or via an API of another organization or entity) using the vaccination identifier collected from the scannable identifier. The scannable identifier can be matched against a record at a data repository, data service, or remote database. Upon request, the third party system may return the requested information to the vaccination record system 128. The information received for the vaccine dose may include, but is not limited to, the time of creation, the time of shipping, the time of dispensing, the time of administration, the details of the manufacturer including address and contact information, a manufacturer code, a provider code associated with a provider of the vaccination dose, a provider location of the vaccination dose, and other information. In some embodiments, the vaccination record system 128 may request the vaccination information from more than one location. As an example, the vaccination record system 128 may query the manufacturer for information about where the vaccine dose was shipped and a vaccine registry for when the vaccine dose was distributed. The information can include location where the vaccine was created, the location of the vaccine's distributer, the location of the provider, the date the vaccine was created, when it was shipped, and when the vaccine was bottled. In some embodiments, some of the information can be stored in a scannable identifier (e.g., a QR code or RFID tag).

At box 3415, the process 3400 can include receiving a provider confirmation code (or provider code) corresponding to a provider. The transfer of the provider confirmation code to the mobile phone 108 may be done wirelessly, over internet communication (email or application communication), or through other scanning processes. In some embodiments, the provider code can be provided by the provider at the vaccination site. The provider may tell the patient the provider code, submit the provider code to the vaccination record system 128, provide a scannable identifier corresponding to the provider code (e.g., a QR code, bar code, RFID tag, or other scannable identifier). In some embodiments, the provider can access the vaccination record system 128 to submit the provider code for the vaccination. In one embodiment, the provider can submit the patient information to the vaccination record system 128 to confirm the vaccination was given to the patient. The provider code can correspond to a particular office of the provider such that each office for a single provider has a different provider confirmation code. The provider code can correspond to a single provider such that all provider offices use a same provider code. In some embodiments, the provider code is provided by a third party system. The provider can request a provider code, which can be provided to the patient or the vaccination record system 128. In some embodiments, the provider can generate, via the third party system or by the provider, a new provider code for each transaction or vaccination dose. In some embodiments, a nurse or doctor at the provider's office can enter the provider code.

In some embodiments, the mobile phone 108 can determine the appointment time from a calendar of the user on the mobile phone 108. In other embodiments, the user can enter the appointment time into the mobile phone 108. In one embodiment, the provider confirmation code may include information regarding appointment time of the user, which may help the verification process to be described herein.

At step 3418, the process 3400 can include determining if the collected information matches the expected values to verify the vaccination. As an example, the vaccination record system 128 can determine if the scannable identifier matches a vaccination dose. The vaccination record system 128 can determine if the provider code matches a known provider. The vaccination record system 128 can determine if the vaccination dose was delivered to the provider corresponding to the provider code. The vaccination record system 128 can determine if the appointment time matches the time the vaccination dose was administered. The vaccination record system 128 may validate an appointment time based on the appointment time being within a threshold time window from when the vaccination dose was determined to be administered. The vaccination record system 128 can determine whether the location of the vaccine dose matches an office of the provider. The vaccination record system 128 can determine whether the location of the provider and/or the location of the vaccine dose matches a GPS location of the mobile phone 108 within a time window around the vaccination time or appointment time. The vaccination record system 128 can determine if the confirmation code corresponding to a provider matches the record values for that provider. The vaccination record system 128 can verify the providers location with the location of the confirmation code, verify the location corresponding to the business location of the provider, verify the vaccination information corresponding to the provider, and verify the appointment time of the user 102 with respect to the providers records. This timing system may have a threshold to determine accurate distinctions between scan time and appointment time of the user 102. If the verification is successful, the process 3400 can continue to box 3421. If the verification fails, the process 3400 can continue to the step 3424.

At step 3424, the process 3400 can include determining if the vaccine information can be fixed. For example, if the scannable identifier does not match a known vaccination dose, the vaccination record system 128 may prompt a user to reenter the scannable identifier. As another example, the vaccination record system 128 may request the user or provider to reenter the provider code if the provider code does not match. In some embodiments, the vaccination record system 128 can enable the user to escalate the failure of data to match. By escalating, an administrator can verify information from the user (e.g., that the provider opened a new office that is not yet associated with the provider's account). The administrator can verify and override the failed verification. It can be appreciated that the verification and correction of data can be performed piecemeal as the data is received from the user. As an example, the provider code can be verified when entered and the provider can reenter the provider code while the user is at the office rather than waiting until all information is collected. If the vaccine data can be fixed, the process may return to the decision sate 3418 and reattempt the verification process. If the vaccine data cannot be changed, the process 3400 may end with a failed verification of the provider's confirmation data.

At box 3421, the process 3400 can include associating a vaccination dose with the user account. In one or more embodiments, the user's mobile phone 108 and/or the vaccination record system 128 may store a local association of the vaccination dose with the user's account. In various embodiments, a subset of the collected data associated with the verification process may also be stored in the vaccination record system 128, such as for example, in the user's cloud vault database 126. The vaccination record system 128 can store the data in an encrypted format, such as encryption at rest. The vaccination record system 128 can write the data to a blockchain ledger.

The vaccination record system 128 can facilitate the distribution of confirmation codes by the user 102, such as a certificate of immunization or a health pass. The vaccination record system 128 may generate and transmit any records necessary to indicate that the user has received the vaccination dose. A mobile phone 108 can transfer the record wirelessly or via a code/scannable identifier. For example, the mobile phone 109 can transfer the record via Bluetooth, NFC, or other protocol. The vaccination record system 128 can send the record to a remote device. The remote device can include an electronic medical records (EMR) system of a healthcare provider. The vaccination record system 128 may embed a link to information about the vaccination in a scannable identifier (e.g., a QR code).

In some embodiments, an entity, such as a vaccination registry or a data repository, may track vaccination information. The entity may receive data to add the vaccination to a data store in a predefined data format via an API. The vaccination record system 128 may format the data in the predefined data format and transmit the record to the entity via the API. In some embodiments, the data is de-identifier prior to transmission to prevent violation of privacy laws. In other embodiments, the data is not de-identified, such as if privacy laws are not a concern when sending to the CDC or SIR of a state. The vaccination record system 128 can transmit the vaccination information for one or more users to local, state, or other registries to maintain a vaccination record. Process 3400 may end once the process box 3421 or 3424 are complete or may repeat for another vaccination.

Although the flowchart of FIG. 34 show a specific order of execution, it is understood that the order of execution may differ from that which is depicted. For example, the order of execution of two or more blocks may be scrambled relative to the order shown. Also, two or more blocks shown in succession in FIG. 34 may be executed concurrently or with partial concurrence. Further, in some embodiments, one or more of the blocks/boxes/steps shown in FIG. 34 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

While embodiments of the invention have been particularly shown and described with references to preferred embodiments thereof, in light of the present disclosure it will be understood by persons skilled in the art that various changes in form and details may be made therein without departing from the scope of the invention encompassed by the appended claims.

From the foregoing, it will be understood that various aspects of the processes described herein are software processes that execute on computing devices including computer systems that form parts of the system. The computing devices can include hardware processors and memory devices among other components. Accordingly, it will be understood that various embodiments of the system described herein are implemented as specially-configured computers including various computer hardware components and, in many cases, significant additional features as compared to conventional or known computers, processes, or the like, as discussed in greater detail herein. Embodiments within the scope of the present disclosure also include computer-readable media for carrying or having computer-executable instructions or data structures stored thereon. Such computer-readable media can be any available media which can be accessed by a computer, or downloadable through communication networks. By way of example, and not limitation, such computer-readable media can comprise various forms of data storage devices or media such as RAM, ROM, flash memory, EEPROM, CD-ROM, DVD, or other optical disk storage, magnetic disk storage, solid state drives (SSDs) or other data storage devices, any type of removable non-volatile memories such as secure digital (SD), flash memory, memory stick, etc., or any other medium which can be used to carry or store computer program code in the form of computer-executable instructions or data structures and which can be accessed by a computer, special purpose computer, specially-configured computer, mobile device, etc.

When information is transferred or provided over a network or another communications connection (either hardwired, wireless, or a combination of hardwired or wireless) to a computer, the computer properly views the connection as a computer-readable medium. Thus, any such a connection is properly termed and considered a computer-readable medium. Combinations of the above should also be included within the scope of computer-readable media. Computer-executable instructions comprise, for example, instructions and data which cause a computer, special purpose computer, or special purpose processing device such as a mobile device processor to perform one specific function or a group of functions.

Those skilled in the art will understand the features and aspects of a suitable computing environment in which aspects of the disclosure may be implemented. Although not required, some of the embodiments of the claimed inventions may be described in the context of computer-executable instructions, such as program modules or engines, as described earlier, being executed by computers in networked environments. Such program modules are often reflected and illustrated by flow charts, sequence diagrams, exemplary screen displays, and other techniques used by those skilled in the art to communicate how to make and use such computer program modules. Generally, program modules include routines, programs, functions, objects, components, data structures, application programming interface (API) calls to other computers whether local or remote, etc. that perform particular tasks or implement particular defined data types, within the computer. Computer-executable instructions, associated data structures and/or schemas, and program modules represent examples of the program code for executing steps of the methods disclosed herein. The particular sequence of such executable instructions or associated data structures represent examples of corresponding acts for implementing the functions described in such steps.

Those skilled in the art will also appreciate that the claimed and/or described systems and methods may be practiced in network computing environments with many types of computer system configurations, including personal computers, smartphones, tablets, hand-held devices, multi-processor systems, microprocessor-based or programmable consumer electronics, networked PCs, minicomputers, mainframe computers, and the like. Embodiments of the claimed invention are practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing various aspects of the described operations, which is not illustrated, includes a computing device including a processing unit, a system memory, and a system bus that couples various system components including the system memory to the processing unit. The computer will typically include one or more data storage devices for reading data from and writing data to. The data storage devices provide nonvolatile storage of computer-executable instructions, data structures, program modules, and other data for the computer.

Computer program code that implements the functionality described herein typically comprises one or more program modules that may be stored on a data storage device. This program code, as is known to those skilled in the art, usually includes an operating system, one or more application programs, other program modules, and program data. A user may enter commands and information into the computer through keyboard, touch screen, pointing device, a script containing computer program code written in a scripting language or other input devices (not shown), such as a microphone, etc. These and other input devices are often connected to the processing unit through known electrical, optical, or wireless connections.

The computer that effects many aspects of the described processes will typically operate in a networked environment using logical connections to one or more remote computers or data sources, which are described further below. Remote computers may be another personal computer, a server, a router, a network PC, a peer device or other common network node, and typically include many or all of the elements described above relative to the main computer system in which the inventions are embodied. The logical connections between computers include a local area network (LAN), a wide area network (WAN), virtual networks (WAN or LAN), and wireless LANs (WLAN) that are presented here by way of example and not limitation. Such networking environments are commonplace in office-wide or enterprise-wide computer networks, intranets, and the Internet.

When used in a LAN or WLAN networking environment, a computer system implementing aspects of the invention is connected to the local network through a network interface or adapter. When used in a WAN or WLAN networking environment, the computer may include a modem, a wireless link, or other mechanisms for establishing communications over the wide area network, such as the Internet. In a networked environment, program modules depicted relative to the computer, or portions thereof, may be stored in a remote data storage device. It will be appreciated that the network connections described or shown are exemplary and other mechanisms of establishing communications over wide area networks or the Internet may be used.

While various aspects have been described in the context of a preferred embodiment, additional aspects, features, and methodologies of the claimed inventions will be readily discernible from the description herein, by those of ordinary skill in the art. Many embodiments and adaptations of the disclosure and claimed inventions other than those herein described, as well as many variations, modifications, and equivalent arrangements and methodologies, will be apparent from or reasonably suggested by the disclosure and the foregoing description thereof, without departing from the substance or scope of the claims. Furthermore, any sequence(s) and/or temporal order of steps of various processes described and claimed herein are those considered to be the best mode contemplated for carrying out the claimed inventions. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed inventions. In addition, some steps may be carried out simultaneously, contemporaneously, or in synchronization with other steps.

Embodiments and examples of the invention have been described for the purpose of illustration. Persons skilled in the art will also recognize from this description that the described embodiments and examples are not limiting, and may be practiced with modifications and alterations limited only by the spirit and scope of the appended claims which are intended to cover such modifications and alterations, so as to afford broad protection to the various embodiments of the invention and their equivalents.

What is claimed is:

1. A system, comprising:
a data store; and
at least one computing device in communication with the data store, the at least one computing device configured to:
authenticate a user account associated with a user;
scan an area to determine a scannable identifier associated with a vaccination dose;
determine a batch number and a lot number associated with the vaccination dose based on the scannable identifier;
determine a location associated with the at least one computing device;
receive a provider confirmation code corresponding to a provider;
perform a verification that the provider confirmation code matches an assigned provider confirmation code corresponding to the provider, the location corresponds to a business location of the provider, and the batch number and the lot number associated with the vaccination dose are assigned to the provider; and
in response to the verification being successfully performed, associate the vaccination dose with the user account in the data store as having been given to the user.

2. The system of claim 1, wherein the at least one computing device is further configured to determine an appointment time of the user with the provider.

3. The system of claim 1, wherein the provider confirmation code is further associated with the business location of the provider and a plurality of different provider confirmation codes are individually associated with a respective one of a plurality of other business locations of the provider.

4. The system of claim 1, wherein the at least one computing device is further configured to:
request vaccination information associated with the vaccination dose from a third-party system based on the batch number and the lot number; and
receive the vaccination information from the third-party system.

5. The system of claim 4, wherein the vaccination information from the third-party system comprises a time corresponding to when the vaccination dose was dispensed.

6. The system of claim 1, wherein the area scanned to determine the scannable identifier comprises a vile of the vaccination dose.

7. A method, comprising:
scanning, via at least one computing device, a vial of a vaccination dose to determine a scannable identifier associated with the vaccination dose;
determining, via the at least one computing device, a batch number and a lot number associated with the vaccination dose based on the scannable identifier;
determining, via the at least one computing device, a location associated with the at least one computing device;
receiving, via the at least one computing device, a provider confirmation code corresponding to a provider;
performing, via the at least one computing device, a verification that the provider confirmation code matches an assigned provider confirmation code corresponding to the provider, the location corresponds to a business location of the provider, and the batch number and the lot number associated with the vaccination dose are assigned to the provider; and
in response to the verification being successfully performed, associating, via the at least one computing device, the vaccination dose with a user account as having been given to a user corresponding to the user account.

8. The method of claim 7, wherein associating the vaccination dose with the user account comprises:
generating a record stored locally on the at least one computing device, the record comprising information about the vaccination dose and the user account; and
transmitting the record to a remote data service for storage.

9. The method of claim 8, further comprising:
generating, via the at least one computing device, a second scannable identifier encoding information regarding the record; and
rendering, via the at least one computing device, the second scannable identifier on a display.

10. The method of claim 8, wherein the second scannable identifier comprises a link to a user interface comprising a certificate of immunization for the vaccination dose.

11. The method of claim 8, further comprising:
converting, via the at least one computing device, the record into a predetermined format associated with a third-party; and
pushing, via the at least one computing device, the record in the predetermined format to the third-party.

12. The method of claim 7, further comprising:
requesting, via the at least one computing device, vaccination information associated with the vaccination dose from a third-party system based on the batch number and the lot number; and
receiving, via the at least one computing device, the vaccination information from the third-party system.

13. The method of claim 12, wherein the vaccination information from the third-party system comprises the assigned provider confirmation code corresponding to the provider.

14. A non-transitory computer-readable medium embodying a program that, when executed by at least one computing device, causes the at least one computing device to:
scan a vial of a vaccination dose to determine a scannable identifier associated with the vaccination dose;
determine a batch number and a lot number associated with the vaccination dose based on the scannable identifier;
determine a location associated with the at least one computing device;
receive a provider confirmation code corresponding to a provider;
perform a verification that the provider confirmation code matches an assigned provider confirmation code corresponding to the provider, the location corresponds to a business location of the provider, and the batch number and the lot number associated with the vaccination dose are assigned to the provider; and
generate a record comprising information about the vaccination dose and a user account associated with a user, the record indicating that the user has been given the vaccination dose.

15. The non-transitory computer-readable medium of claim 14, wherein the program further causes the at least one computing device to:
request vaccination information associated with the vaccination dose from a third-party system based on the batch number and the lot number; and
receive the vaccination information from the third-party system.

16. The system of claim 15, wherein the vaccination information from the third-party system comprises the business location of the provider.

17. The non-transitory computer-readable medium of claim 14, wherein the program further causes the at least one computing device to transfer the record indicating that the user has been given the vaccination dose to a remote device.

18. The non-transitory computer-readable medium of claim 17, wherein the remote device comprises an electronic medical records (EMR) system of a healthcare provider.

19. The non-transitory computer-readable medium of claim 17, wherein the record is transferred wirelessly from the at least one computing device using at least one of near field communication (NFC) or Bluetooth.

20. The non-transitory computer-readable medium of claim 14, wherein the record comprises a digital signature generated by a particular certificate authority.
